(19) United States
(12) Patent Application Publication
(10) Pub. No.: US 2019/0201183 A1
(43) Pub. Date: Jul. 4, 2019

(54) TISSUE ATTACHMENT DEVICE AND METHOD

(71) Applicant: Zift, LLC, Minnetrista, MN (US)

(72) Inventors: Robert S. Schwartz, Inver Grove Heights, MN (US); Stanton J. Rowe, Newport Coast, CA (US); Robert A. Van Tassel, Minnestrista, MN (US); Ralph Schneider, Trabuco Canyon, CA (US); Ming Wu, Tustin, CA (US); David John Blaeser, Brooklyn Park, MN (US); Eric J. Simso, Minnetrista, MN (US); Philip Jon Haarstad, Chanhassen, MN (US); Douglas Jay Krone, Rogers, MN (US); Brian Zelickson, Minneapolis, MN (US); Robert A. Ganz, Minnetonka, MN (US); Matt Blaeser, Brooklyn Park, MN (US); Matt Keillor, Inver Grove Heights, MN (US)

(73) Assignee: Zift, LLC, Minnetrista, MN (US)

(21) Appl. No.: 15/858,091

(22) Filed: Dec. 29, 2017

(51) Int. Cl. A61F 2/08; A61F 2/10; A61B 17/064

(52) U.S. Cl. CPC A61F 2/0811; A61F 2/105; A61B 17/0642; A61B 17/0644; A61F 2002/0817; A61B 2017/00867; A61F 2002/0888; A61F 2220/0016; A61B 2017/0641; A61B 2017/0648; A61F 2002/0864

(57) ABSTRACT

A method and apparatus for attaching tissue to bone in a shifted position without requiring surgical detachment of muscle or connective tissue joining the tissue layer to the bone layer. The skin and/or soft tissue layer is displaced in a surgical or non-surgical manner and a fastener of the invention is driven through the skin and/or soft tissue layer into the periosteum or bone layer to affect a tissue repositioning procedure. The method and apparatus may also be used to attach tissue to tissue an a shifted position.

### RELATED APPLICATIONS

This application incorporates by reference U.S. application Ser. No. 13/961,785 filed Aug. 7, 2013 entitled Tissue Attachment Device And Method.

### FIELD OF THE INVENTION

This invention generally relates to a method and apparatus for tissue attachment, with one or more tissue layers being attached to bone or other tissue layers. There are multiple applications, including but not limited to dermatology/plastic surgery among others. The application specifically described in this disclosure is to reposition the skin and/or surrounding tissues, of the face or any other body part in relationship to associated bone, cartilage or tissue structures, then secure the tissue in the new position. The overall effect is to restore tissue to a preferred position, such as might be done in a brow-lift or other cosmetic procedure or in orthopedic procedures such as tendon reattachment.

### BACKGROUND OF THE INVENTION

The current endoscopic, surgical or open facelift procedure (also known as a rhytidectomy) involves the surgical removal of excess skin and tissue from the face and the redraping of the remaining skin of the face. The open procedure involves making an incision from below the ear, which extends up into the hairline across the top of the hairline and around to bottom of the ear on the opposite side. After the incision is made, the skin and/or underlying tissues are separated from the bone and or periosteum using blunt dissection, t, and the skin redraped and sutured in the new position, with the excess skin being removed. A variation of this procedure is the endoscopic facelift in which a series of smaller incisions (10-30 mm) are made at or above the hairline, and using blunt dissection and endoscopic guidance the tissue is released from the underlying bone. A variety of techniques such as sutures, screws, bone anchors, bone tunnels and various implants are used to elevate the tissue and secure the tissue to the underlying bone. An even more limited approach is the Brow-pexy in which small (1-2 cm) incisions are made below the brow, and blunt dissection is again used to separate the soft tissue from underlying bone and various means are used to secure the soft tissue in an elevated position.

All variations of aesthetic procedures are expensive, involve a long and painful recovery period, and may be accompanied by complications such as infection, bleeding, nerve damage and complications from anesthesia.

Efforts have been directed lately toward various techniques that involve fixation of skin through the use of permanent or resorbable tacks or anchors to effect facial rejuvenation. However, these procedures still involve the use of significant incisions, requiring a substantial recovery period, and associated surgical, anesthesia, and post-surgical risks. There is thus a need in dermatology and plastic surgery for a less invasive, less expensive method for reducing the effects of ageing and restoring tissue to it's natural position on the face and other areas of the body. Such a method would also have utility in the treatment of facial paralysis due to stroke, Bells Palsy, or surgical or other trauma. The procedure might also have utility in treating patients with traumatic injuries to the face such as might be caused by automobile accidents or battlefield injuries. The procedure might also have utility in treating common tendon avulsion injuries such as mallet or baseball finger.

Methods and devices that addresses the above need has been developed and are shown and described in U.S. application Ser. No. 13/961,785 filed Aug. 7, 2013 entitled Tissue Attachment Device And Method, incorporated by reference herein in its entirety. The methods and devices are generally directed to accomplishing the steps of moving the soft tissue layer to a new position in relation to the bone or cartilage below; having a device which is either pushed or ballistically and dynamically driven into the deep layer of bone, cartilage or soft tissue through a superficial soft tissue layer, thereby holding the superficial soft tissue layer in the new position in relation to the bone, cartilage or soft tissue through the use of anchors.

Prior to the development of this technology, little or no experimentation had been performed in the area of ballistically firing micro-pins into the tissue/bone layers of the human skull. Since the filing of the aforementioned application, much has been learned about the dynamics of firing these micro-pins into a variety of bones, resulting in variations in methods and designs in both the pins and delivery devices. At least some of these variations are described herein.

### SUMMARY OF THE INVENTION

Several embodiments are shown and described herein directed to percutaneously lifting, translating, and repositioning the skin and/or underlying tissues. There are several applications in which the methods and devices of the invention could be applied.

For example, the methods and devices of the invention are useful in facial procedures, such as effecting a reduction in the appearance of wrinkles and excess skin on the face and other areas of the body an/or the translation of the skin and/or underlying tissues in order to correct the position of the skin and associated anatomical features. In one application, the device could be used to reposition the brow to help correct for brow ptosis and/or visual field impairment. Another example would be to correct for facial asymmetry caused by muscle paralysis due to stroke or Bell's palsy. Another example would be to correct facial deformities due to trauma of various kinds. The method of the present invention enables a minimally invasive procedure for repositioning the skin and associated anatomical features.

The methods and devices of the invention are useful in other, non-facial applications as well. For example, the devices and methods are well-suited for reattaching tendons, such as in the hand or other areas.

More generally, one aspect of the invention provides a method for relocating a skin and/or underlying tissues layer relative to an adjacent bone layer comprising: shifting a skin and/or underlying tissues layer relative to an adjacent bone layer from an original position to a shifted position without cutting deep muscle or connective tissue associated with the skin layer; holding the skin layer and underlying tissues in the shifted position while driving a fastener through the skin layer into the bone layer a desired depth to prevent the skin layer and/or underlying tissues from reassuming the original position; wherein driving the fastener through the skin layer into the bone layer comprises transferring a minimum amount of energy to the fastener.

Transferring a minimum amount of energy to the fastener may comprise accelerating the fastener to a minimum velocity. The minimum velocity may be determined by a size of the fastener. Alternatively, the minimum velocity may be determined by a size of a predrilled hole in the bone relative to a size of the fastener. The size of the predrilled hole may comprise a depth of the predrilled hole. The size of the predrilled hole may comprise a width of the predrilled hole.

Transferring a minimum amount of energy to the fastener may be accomplished by accelerating the fastener with a firing pin.

The depth the fastener is driven into the bone layer may be controlled by a travel length of the firing pin or by applying a control level of energy to the fastener.

Another aspect of the invention provides an implant for use in anchoring a skin layer and/or underlying tissues in a shifted position to a bone layer thereby overcoming lateral and axial forces imparted by the skin layer and/or underlying tissues on the implant comprising: a fastener having a hollow shaft with a distal end and a proximal end, the hollow shaft including: an anchoring feature proximate the distal end; and, a tissue-holding feature proximate the proximal end; and a spike housed within the hollow shaft and protruding from a distal end thereof, the spike including a sharpened tip.

In one aspect of the invention, the method includes the introduction of a substance, for example an adhesive (e.g. fibrin glue) to bond the device to the bone or bond the tissue to the bone. Additionally or alternatively, the substance could be one that promotes healing. The substance could be introduced via the delivery device or through the implant itself, either through the implant or applied to the implant as a coating.

Another aspect of the invention provides an implant for use in anchoring a skin layer and/or underlying tissues in a shifted position to a bone layer thereby overcoming lateral and axial forces imparted by the skin layer and/or underlying tissues on the implant comprising: a fastener having a hollow shaft with a distal end that includes a sharpened leading edge and a proximal end, the hollow shaft including: an anchoring feature proximate the distal end; and, a tissue-holding feature proximate the proximal end.

The tissue-holding feature may comprise at least one petal that flares outwardly upon deployment. At least one petal may comprise a memory metal.

The anchoring feature may comprise at least one barb that flares outwardly upon deployment.

The anchoring feature may comprise at least one petal that flares outwardly upon deployment

The distal end of the hollow shaft may comprise a bevel that aligns with a surface of the sharpened tip.

The sharpened tip may comprise a flange that has a diameter greater than a diameter of the hollow shaft.

In some applications, the tissue-holding ability of the device may be enhanced by forming a dissection plane in the targeted tissue. The dissection plane is formed by releasing underlying tissue to induce scarring, thereby taking advantage of the increased durability that scar tissue provides. We have contemplated releasing the underlying tissues sub-periosteally or super-periosteally through the same or a different small incision prior to placement of anchors. This can be accomplished utilizing a small tool, such as a blunt dissecting tool or elevator, that would be placed through the small incision.

Implantation can be accomplished super-periosteally.

Another aspect of the invention provides an implant for use in anchoring a skin layer and/or underlying tissues in a shifted position to a bone layer thereby overcoming lateral and axial forces imparted by the skin layer and/or underlying tissues on the implant comprising: a fastener having a hollow shaft with a distal end and a proximal end, the hollow shaft including: a tissue-holding feature proximate the proximal end; and a screw housed within the hollow shaft and protruding from a distal end thereof.

The tip of the screw may comprise as self tapping feature at its distal end. Alternatively or additionally, a hole may be pre-drilled in the bone. It is likely that the size of the screw for a given application may dictate whether pre-drilling is merited.

Another aspect of the invention provides an implant for use in anchoring a skin layer and/or underlying tissues in a shifted position to a bone layer thereby overcoming lateral and axial forces imparted by the skin layer and underlying tissues on the implant comprising: a fastener having a screw like element with a proximal and distal end. The proximal end may have a section of increased diameter to interact with a tissue holding feature. A tissue holding feature that is configured to accept a screw like feature.

The tissue holding feature may be made from a flat sheet or hollow tube.

Another aspect of the invention provides an implant for use in anchoring a skin layer and/or underlying tissues in a shifted position to a bone layer thereby overcoming lateral and axial forces imparted by the skin layer and/or underlying tissues on the implant comprising: a fastener fixed to the bone and a tissue holding feature that is displaced laterally some distance away from the fastener.

Another aspect of the invention provides an implant for use in anchoring a skin layer and/or underlying tissues in a shifted position to a bone layer thereby overcoming lateral and axial forces imparted by the skin layer and/or underlying tissues on the implant comprising: a fastener fixed to periosteal tissue and a tissue holding feature.

Another aspect of the invention provides an implant for use in anchoring a skin layer and/or underlying tissues in a shifted position to a bone layer thereby overcoming lateral and axial forces imparted by the skin layer and/or underlying tissues on the implant comprising: a fastener fixed to periosteal tissue and a tissue holding feature that is displaced laterally some distance away from the fastener.

Another aspect of the invention provides an implant for use in anchoring a skin layer and/or underlying tissues in a shifted position to a bone layer thereby overcoming lateral and axial forces imparted by the skin layer and/or underlying tissues on the implant comprising: a tissue holding feature that is displaced laterally some distance away from another tissue holding feature.

Another aspect of the invention provides an implant for use in anchoring a skin and/or soft tissue layer that includes a tissue holding feature that is adjustably displaced from an anchor and/or from another tissue holding feature. In addition to being adjustable, one embodiment provides a connecting element between the tissue holding feature and the fastener (or another tissue holding feature) that expands and contracts longitudinally. Doing so allows for tissue relaxation over time.

Another aspect of the invention provides a device for use in anchoring a skin layer and/or underlying tissues in a shifted position relative to a bone layer comprising: a firing gun generally including an energy source, a firing pin and an implant.

One embodiment of the device includes a handle; a firing mechanism disposed within the handle and including a firing pin and a trigger mechanism; a disposable tip removably attached to a distal end of the firing gun and including: a proximal tip component that removably attaches the disposable tip to the distal end of the firing gun; a distal tip component distally attached to the proximal tip component; an implant carried within the distal tip component; wherein when the trigger mechanism is pulled: the firing mechanism is activated, thereby transferring energy to the firing pin; the firing pin in turn accelerates the implant to at least a minimum velocity, thereby driving the implant into an implantation site.

The firing mechanism may comprise a spring.

The firing mechanism may comprise a compressed gas.

The firing mechanism may comprise an electronic solenoid or similar electromechanical feature.

The firing mechanism may comprise a hydraulic fluid.

The proximal tip component may slidingly attach to the distal tip component.

The safety feature may require the firing gun to be compressed against a surface in order to activate the triggering mechanism.

Another aspect of the invention provides a fastener for use in holding a tissue layer in a shifted position relative to an adjacent tissue layer. The fastener includes a tissue holder for holding the shifted layer and an anchor for fixing the fastener relative to the adjacent tissue layer, such as the periosteum or bone layer. The tissue holder is connected to the anchor with a shaft that runs substantially parallel to the adjacent tissue layer. The lateral displacement between the tissue holder and the anchor or anchors may be variable. Additionally, there may be one or more tissue holders positioned either adjacent to each other, or laterally displaced from each other. The tissue holders may be round, oblong or angled.

The anchor may include one or more barb or other fasteners such as spikes, screws or tacks. The anchors may be designed to engage any soft tissue, such as the periosteum, or the bone layer.

The fastener may be constructed of a memory metal, such as nitinol, such that the tissue holder may transform from a straight, delivered configuration, to a curled deployed configuration upon release from a delivery device. The tissue holder, anchor, or both may be constructed from a biodegradable/bioabsorbable material.

The delivery device may be a low profile catheter or cannula from which the fastener is ejected during delivery. The fastener may be loaded into the delivery device such that the anchor side of the fastener is distal and emerges first, or it may be loaded such that the tissue holder is distal and emerges first.

### BRIEF DESCRIPTION OF THE DRAWINGS

These and other aspects, features and advantages of which embodiments of the invention are capable of will be apparent and elucidated from the following description of embodiments of the present invention, reference being made to the accompanying drawings, in which:

FIG. 1a is a step in a method of the invention to relocate tissue;

FIG. 1b is a step in a method of the invention to relocate tissue;

FIG. 1c is a step in a method of the invention to relocate tissue;

FIG. 2 is a cutaway view of a fastener of the present invention embedded into tissue;

FIG. 3 is a perspective view of an embodiment of an implant of the invention;

FIG. 4 is a perspective view of an embodiment of a spike of the invention;

FIG. 5 is a perspective view of an embodiment of an implant of the invention;

FIG. 6 is a perspective view of an embodiment of a spike of the invention;

FIG. 7a is a perspective view of an embodiment of an implant of the invention in a delivery configuration;

FIG. 7b is a perspective view of the embodiment of FIG. 7a in a deployed configuration;

FIG. 8 is a depiction of a step of an implantation procedure of the invention;

FIG. 9 is a depiction of a step of an implantation procedure of the invention;

FIG. 10 is a depiction of a step of an implantation procedure of the invention;

FIG. 11 is a depiction of a step of an implantation procedure of another embodiment of the invention;

FIG. 12 is an elevation of an embodiment of a fastener of the invention;

FIG. 13 is a perspective view of the fastener of FIG. 12 implanted on a patient;

FIG. 14 is a close-up view of the depiction of FIG. 13;

FIG. 15 is a depiction of an embodiment of a fastener of the invention and its position in relation to skin layers when implanted;

FIG. 16 is an elevation of an anchoring end of an embodiment of a fastener of the invention;

FIG. 17 is a perspective view of an embodiment of a fastener of the invention;

FIG. 18 is an elevation of an embodiment of a fastener of the invention;

FIG. 19 is an elevation of an embodiment of a fastener of the invention;

FIG. 20 is an elevation of an embodiment of a fastener of the invention;

FIG. 21 is an elevation of an embodiment of an anchor of a fastener of the invention;

FIG. 22 is a perspective view of an embodiment of an anchor of a fastener of the invention;

FIG. 23 is an elevation of an embodiment of a fastener of the invention;

FIG. 24 is an elevation of an embodiment of an anchor of a fastener of the invention;

FIG. 25 is a perspective view of an embodiment of an anchor of a fastener of the invention;

FIG. 26 is a depiction of an embodiment of a fastener of the invention and its position in relation to skin layers when implanted;

FIG. 27 is a perspective view of an embodiment of a fastener of the invention;

FIG. 28 is an elevation of the embodiment of FIG. 27 deployed in tissue;

FIG. 29 is a perspective view of an embodiment of a faster of the invention;

FIG. 30 is an elevation of the embodiment of FIG. 29 deployed in tissue; and,

FIG. 31 is a perspective view of an embodiment of a fastener of the invention.

FIG. 32 is a perspective view of an embodiment of a delivery device of the invention;

FIG. 33 is an exploded view of an embodiment of a device of the invention;

FIG. 34 is a cutaway view of a portion of an embodiment of a firing gun of the invention;

FIG. 35a is a closeup of an embodiment of a firing mechanism acting on an embodiment of a fastener of the invention;

FIG. 35b is a closeup of an embodiment of a firing pin of the invention acting on an embodiment of a fastener of the invention;

FIG. 36a is a step in a sequence of steps taken by a firing mechanism of the invention;

FIG. 36b is a step in a sequence of steps taken by a firing mechanism of the invention;

FIG. 36c is a step in a sequence of steps taken by a firing mechanism of the invention;

FIG. 37 is a perspective view of a disposable tip of the invention.

FIG. 38 is a perspective view of an embodiment of a delivery device of the invention;

FIG. 39 is a step in the use of an embodiment of a delivery device of the invention;

FIG. 40 is a step in the use of an embodiment of a delivery device of the invention;

FIG. 41 is a step in the use of an embodiment of a delivery device of the invention;

FIG. 42 is a step in the use of an embodiment of a delivery device of the invention;

FIG. 43 is a step in the use of an embodiment of a delivery device of the invention;

FIG. 44 is a step in the use of an embodiment of a delivery device of the invention; and,

FIG. 45 is a step in the use of an embodiment of a delivery device of the invention.

### DESCRIPTION OF EMBODIMENTS

Specific embodiments of the invention will now be described with reference to the accompanying drawings. This invention may, however, be embodied in many different forms and should not be construed as limited to the embodiments set forth herein; rather, these embodiments are provided so that this disclosure will be thorough and complete, and will fully convey the scope of the invention to those skilled in the art. The terminology used in the detailed description of the embodiments illustrated in the accompanying drawings is not intended to be limiting of the invention. In the drawings, like numbers refer to like elements.

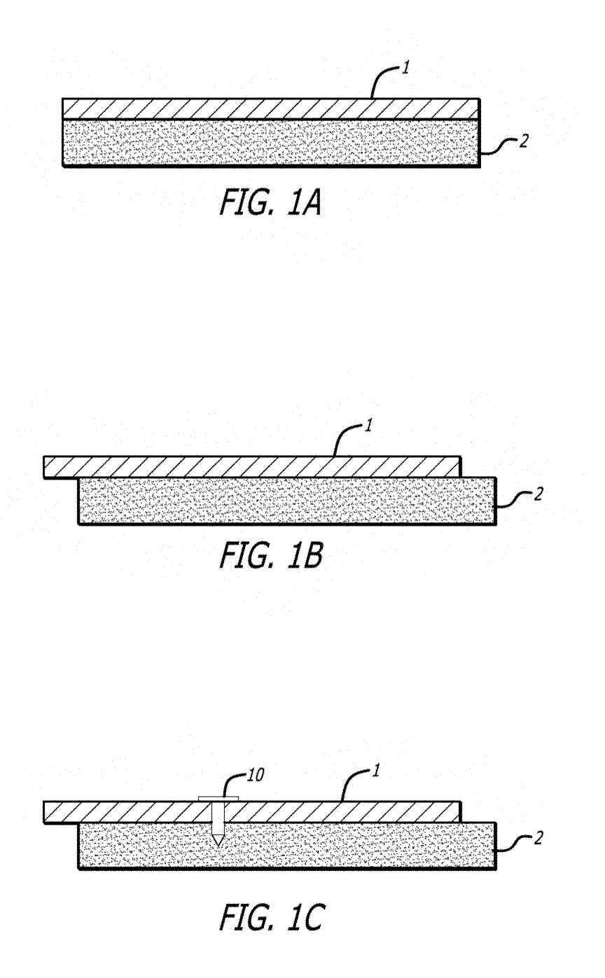

Referring now to the figures, FIG. 1 illustrates the general method of the present invention. As shown in FIG. 1a, the method begins with a selection of a skin layer and/or underlying tissues 1 to be relocated relative to a bone or cartilage layer 2. In FIG. 1b, the skin layer and/or underlying tissues 1 have been shifted relative to the bone layer 2. No connective tissue between the skin layer and underlying tissues 1 and the bone layer 2 have been severed or damaged. The tissue and underlying tissues 1 have simply been relocated using gentle pressure, such as by a finger. Most procedures will attain desirable results with only a small shifting, akin to taking one's finger and moving one's forehead skin around gently. It is noted that the discontinuity in the edges of the tissue sample shown in FIG. 1b are simply provided for illustrative purposes to show that the skin layer and underlying tissues 1 have been shifted relative to the bone layer 2 and is not to be interpreted as a severing or slicing of the selected layer 1. In actuality, the continuous skin layer and underlying tissues 1 is shifted via existing glide planes, and thus releasing the skin layer and underlying tissues 1 would result in its return to the original position shown in FIG. 1a. The method alternatively could begin with blunt dissection of the tissue layer directly above or below the periosteum, releasing the fixation of any or all of the tissue layers above the periosteum relative to the bone.

In order to prevent the return of the skin layer and/or underlying tissues 1 to its original position relative to the bone or cartilage layer 2 of FIG. 1a, a fastener 10 of the present invention is used to affix the skin layer and/or underlying tissues 1 to the bone or cartilage layer 2 at its new position. This step is shown in FIG. 1c. The fastener 10 is shown as a simply tack-like device in FIG. 1c. Various embodiments of fastener 10 will be shown and described below.

The method described above and shown in FIG. 1 is a simple illustration of the basic concepts of the present invention. It is envisioned that the steps of shifting the skin and/or underlying tissues and anchoring it in a new position may be repeated multiple times until a desired result is achieved. It is also likely that a single shift will be followed by multiple fasteners 10 be applied to secure a given shift of the skin layer and/or underlying tissues 1. Subsequent shifts may be required in order to achieve a desired, natural-looking result.

In one embodiment of the method of the present invention, extremely small, short and shallow incisions may be made at the site where the fastener 10 is being driven such that the head or top of the fastener rests just below the surface of the skin such that the fastener remains hidden. These incisions are so small that bleeding is minimal and no incision closure is required. Positive results have been attained with incisions that are no longer than 0.05 inches.

In order to accommodate the insertion of several fasteners 10, various delivery mechanisms are described below, some of which allow the delivery of rapidly successive fasteners, in the spirit of tools found in carpentry, such as nail guns, staple guns, and the like. Other delivery mechanism described below allow for the delivery of several fasteners simultaneously.

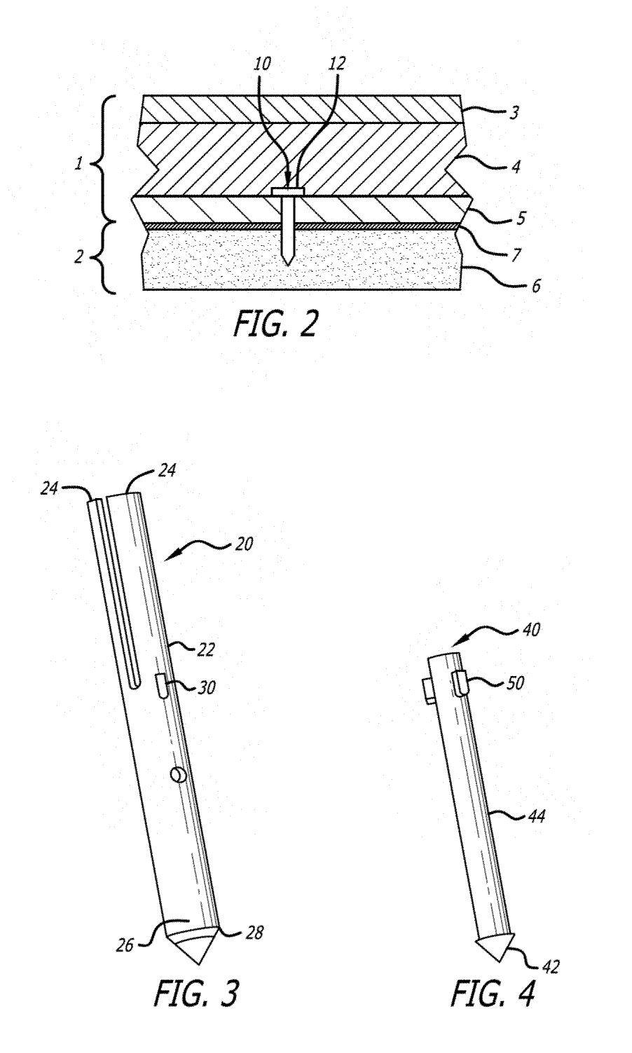

FIG. 2 provides a more detailed view of the skin layer and underlying tissues 1, the bone or cartilage layer 2 and the device 10. The skin layer and underlying tissues 1 comprises an epidermis 3, a dermis 4 and a subcutaneous layer 5. A fibrous tissue layer or fascia (referred to as the superficial musculoaponeurotic system (SMAS) in the face below the eyes and Galea Frontalis in the forehead) is a fanlike structure that envelops the face and provides a suspensory sheet which distributes forces of facial expression. On a cellular level it comprised of collagen fibers, elastic fibers, fat cells and muscle fibers. This fibrous tissue layer resides above the Periosteum or Pericranium.

The bone layer 2 includes the bone 6 and periosteum 7. It can be seen in FIG. 2 that the fastener 10 is implanted such that it penetrates the bone 6 and the periosteum 7 and the proximal end 12 of the fastener 10 terminates within the subcutaneous layer 5, or the dermis 4. In this way, the fastener 10 remains invisible once implanted.

Implants/Fasteners

Turning now to the fasteners of the invention, the fasteners may take one of many possible forms. Generally, they may be circular, flat, or any other configuration geometrically that allows them to penetrate the bone or cartilage with a sharpened distal end. The device may be textured on the surface, for example with a micro-texturing that allows cells to more easily attach and anchor the device permanently in the bone or cartilage. The anchors may be metallic or they may be polymeric. They may be a combination of metal and polymer. The polymer may be biostable or bioabsorbable. The metal may be resorbable such as some magnesium or iron alloys. It may contain drugs for elution. The anchors may be electrically conductive and may permit electrical energy for either energy delivery or energy recording of biologic signals. Examples of embodiments having various characteristics are shown in the Figures and are not meant to be limiting. It is to be understood that any of the characteristics may be incorporated into any of the embodiments of the invention.

Generally, the fasteners may include one or more shafts and also include an anchor proximate one end of the shaft and a tissue-holding feature, hereinafter “tissue holder,” proximate the other end of the shaft. A first embodiment 20 of an fastener 10 is shown in FIG. 3. This fastener 20 includes a fastener 22 and a spike 40 (FIG. 4) that may be contained within the fastener 22.

The fastener 22 may be a memory metal, such as Nitinol, that includes soft tissue anchoring features that expand or flare outwardly. Alternatively, these features may be made of other materials and flare upon impact with the bone layer (in-situ formation of the feature). The fastener shown in FIG. 3 includes tissue holders 24 and distal anchors 26. The tissue holders 24 are in the form of elongate petals that are formed by cutting slits in the tubular fastener 22. The elongate petals 24 curl outwardly upon deployment, catching the fibrous tissue layer.

The distal anchoring features 26 comprise barbs that flare radially upon implantation. These barbs 26 are generally smaller than the petals 24 as they are designed to imbed themselves in bone rather than soft tissue. The distal anchoring features 26 of FIG. 3 are shown as circumferentially-formed features. It is anticipated that the device may work sufficiently without the distal features (barbs), but that they may aid in long term securement.

The distal end 28 of the fastener 22 may be beveled to match a slope of the distal end of the spike 40, discussed below. Additionally, the fastener 22 may include an aperture 30 for receiving a locking feature, such as a protuberance 50, on the spike 40.

The spike 40 assists in driving the fastener 22 into the bone layer. To accomplish this assistance, the spike 40 includes a sharpened distal end 42 and a solid shaft 44. The spike 40 may be formed of a hardened material such as stainless steel or Titanium. The solid shaft 44 provides column strength, and prevents the hollow shaft of the fastener 20 from bending or otherwise collapsing upon impact with either the driving mechanism or the bone. The spike 40 shown in FIG. 4 includes a locking feature 50 in the form of a groove, notch or protuberance. The locking feature 50 ensures that the spike 40 and the fastener 20 act in unison when being driven into the bone. Alternatively, the spike 40 and fastener 20 could be welded, glued or otherwise attached to each other. Alternatively the spike 40 and fastener 20 could be fashioned from a single piece of material. Alternatively the spike 40 could be temporary and removed after the fastener 20 is in place.

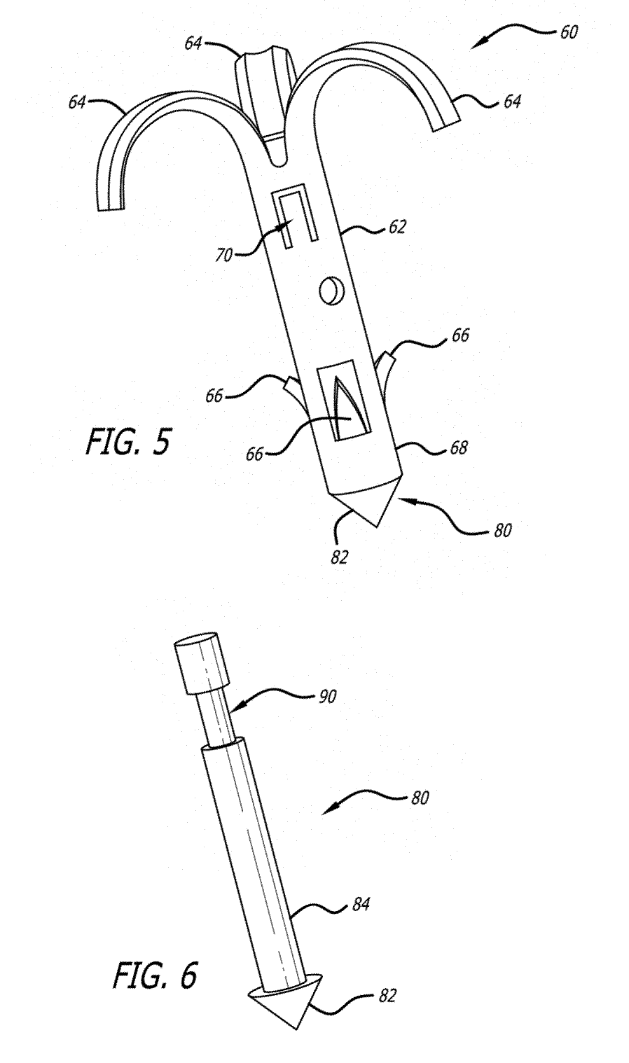

FIGS. 5 and 6 shows another embodiment 60 of a fastener 62 and a spike 80. The fastener 62 includes tissue holders 64 and distal anchors 66. The tissue holders 64 are in the same form of elongate petals as the features 24 shown in FIG. 3, except that the holders 64 are shown in the expanded state. The elongate petals are loaded in a straight configuration but curl outwardly upon deployment, securing the soft tissue of the dermis and/or fibrous tissue layers.

The distal anchors 66 comprise barbs that flare radially upon implantation. These barbs 66 are smaller than the petals 64 as they are design to imbed themselves in bone rather than soft tissue. The distal anchors 66 of FIG. 5 are shown as longitudinally-formed features.

The distal end 68 of the fastener 62 is flat to act against a corresponding feature of the distal end of the spike 80, discussed below. Additionally, the fastener 62 may include a tab locking feature 70 that is inwardly biased to engage a corresponding recess 90 in the spike 80.

The spike 80 assists in driving the fastener 62 into the bone layer. To accomplish this assistance, the spike 80 includes a sharpened distal end 82 and a solid shaft 84. The sharpened distal end 82 is slightly larger than the diameter of the fastener 62. The enlarged end 82 creates a larger hole in the bone, which facilitates the driving of the fastener 62 into the bone material. Because the enlarged end 82 is slightly larger than the diameter of the fastener 62, the end 82 acts as another anchor for the fastener 60.

The spike 80 may be formed of a hardened material such as stainless steel or Titanium. The solid shaft 84 prevents the hollow shaft of the fastener 62 from bending or otherwise collapsing upon impact with either the driving mechanism or the bone. The spike 80 shown in FIG. 6 includes a recess 90 that is engaged by the tab locking feature 70 of the fastener 62. The locking feature 70 ensures that the spike 80 and the fastener 62 act in unison when being driven into the bone.

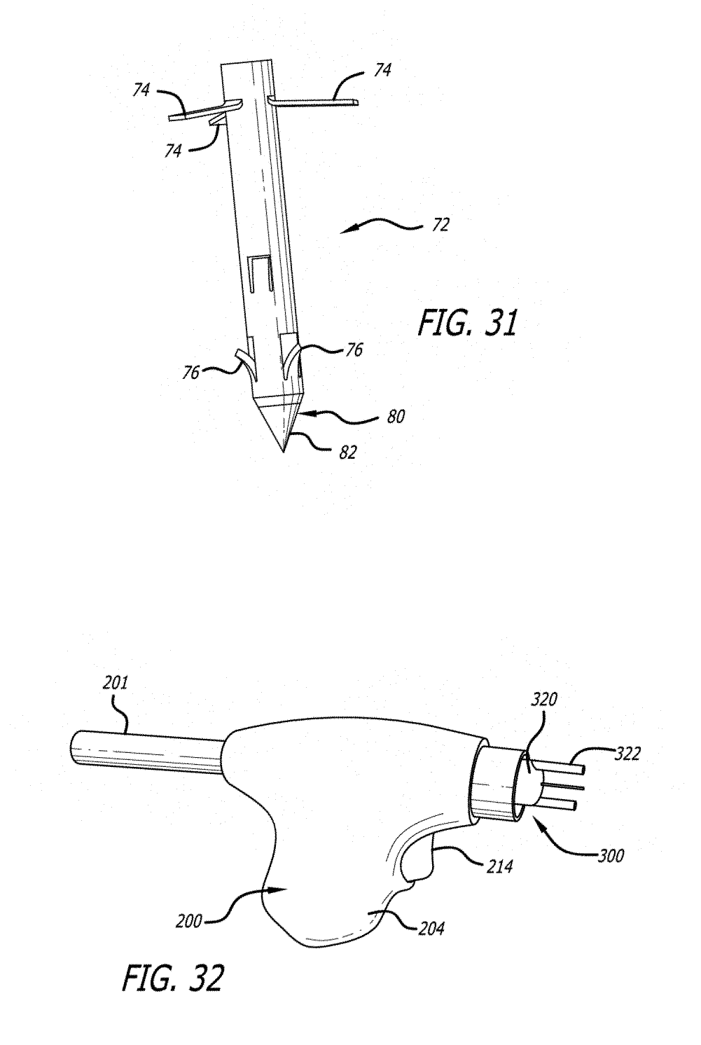

Fastener 62 is shown as having tissue holders 64 that curl distally, toward the anchors 66. However, an embodiment of a fastener 72, shown in FIG. 31, includes tissue holders 74 that flare proximally, away from anchors 76. The fastener 72 may be used with spike 80.

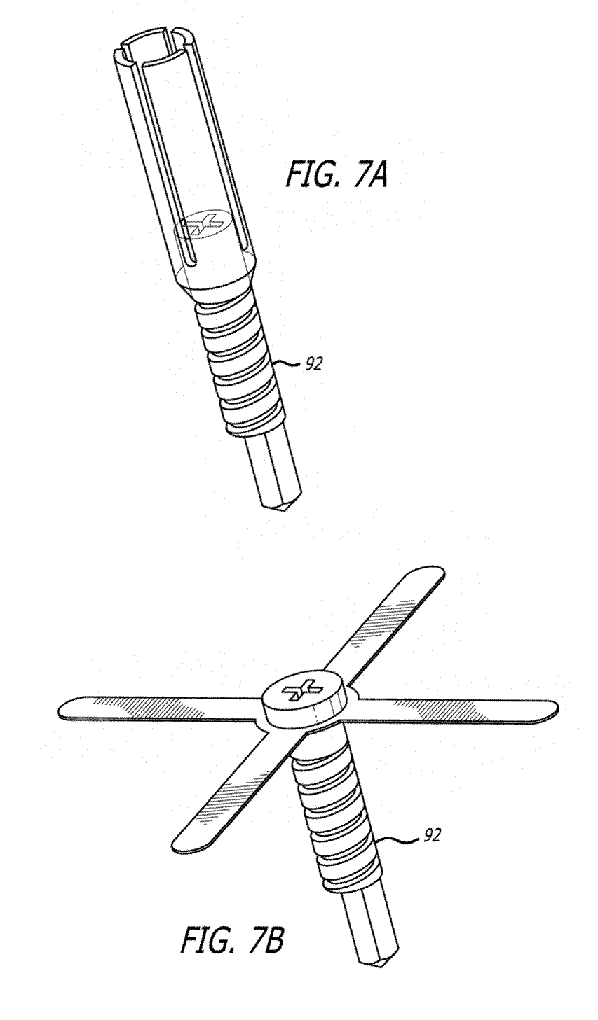

FIGS. 7a and 7b show an alternative spike design in the form of a screw 92. The screw 92 may be used in conjunction with a predrilled hole in the bone or may be self-tapping. The screw 92 is attached to a fastener such that the fastener is carried with the screw 92. FIGS. 7a and 7b show the screw 92 being used in conjunction with a fastener 10.

FIGS. 8-10 show a delivery sequence for a fastener 10. Beginning with FIG. 8, a small incision is made and then a blunt dissection is performed with a cannula 100 down to the surface of the bone layer. The tip 102 of the cannula 100 may have protrusions 104 that slightly penetrate into the bone to aid in fixating the cannula 100 in a desired position relative to the bone.

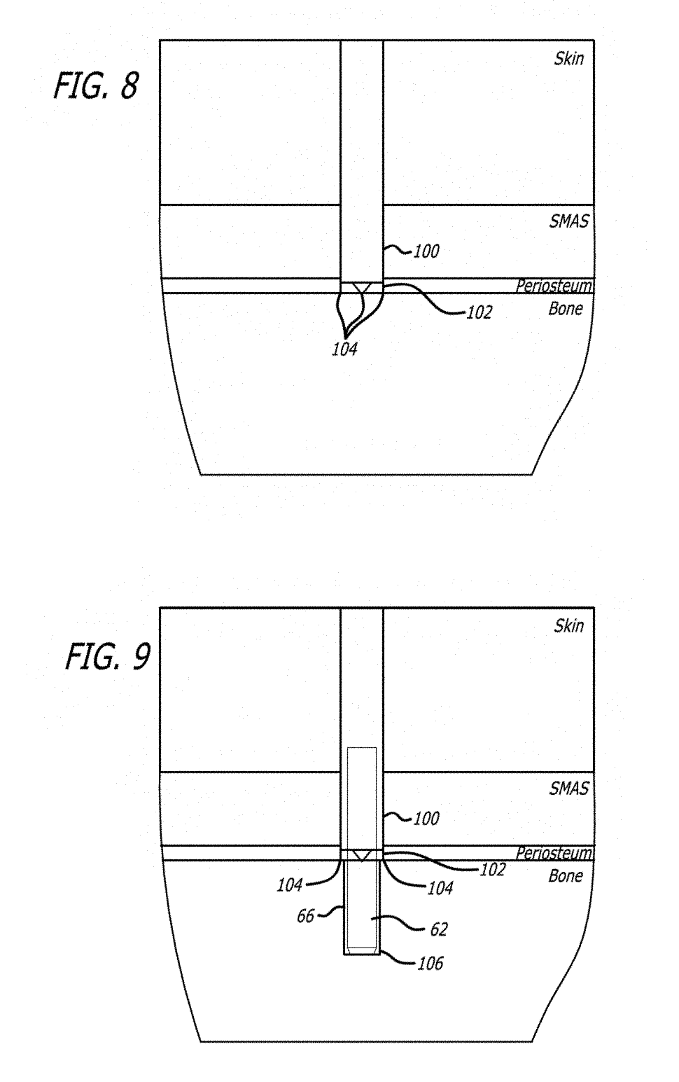

In FIG. 9, a hole 106 is pre-drilled into the bone. The hole is slightly oversized such that the fastener 62 is easily placed into the hole. It can be seen that the fastener 62 has anchors in the form of barbs 66 that flare outwardly to anchor the fastener 62 into the bone. Many of the fastener embodiments described herein have sharpened distal tips, such as those of FIGS. 3-5, and would not require this pre-drilling step, as they are able to be driven directly into the bone.

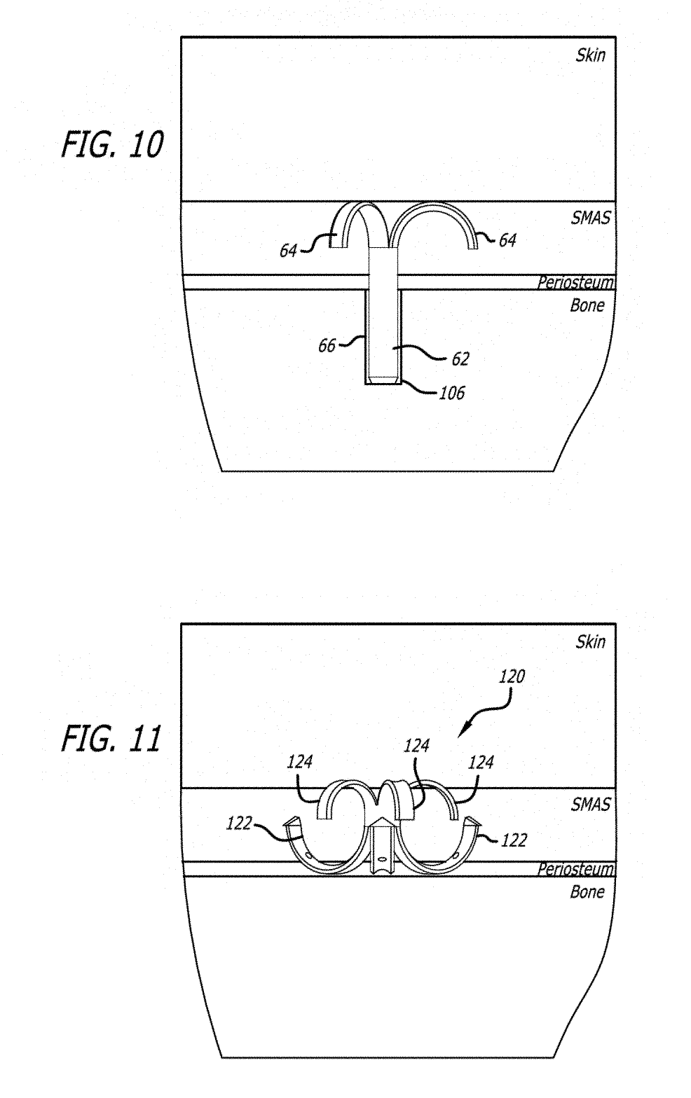

FIG. 10 shows that the cannula 100 has been removed and the fastener 62 remains. The fastener 62 has tissue holders in the form of petals 64 that have curled outwardly, through the soft tissue, thereby capturing the fibrous tissue layer.

FIG. 11 shows a double-petal design for a fastener 120. The fastener 120 has anchors in the form of distal petals 122 and tissue holders in the form of proximal petals 124. The distal petals 122 penetrate only through the periosteum (Pericranium), but do not penetrate into the bone, thus accomplishing fixation by penetrating only the relatively strong and in-elastic pereosteum.

The proximal petals 124 secure the deep dermal tissue and/or fascia layers. This embodiment exhibits greater ease in deployment and removal, if necessary. It has also been demonstrated that these distal petals can be designed to penetrate the bone itself to anchor the device.

It must be emphasized that any of the features described herein with regard to one embodiment may be combined with any of the features of the other embodiments. It is further to be understood that the terms “anchors” and “tissue holders” are being used to distinguish the deeper anchor features from the shallower anchor features. Both the anchors and the tissue holders could be described as functioning as “anchors” or as “tissue holders”. As such the names given to each are not to be interpreted as limiting the functions of the features.

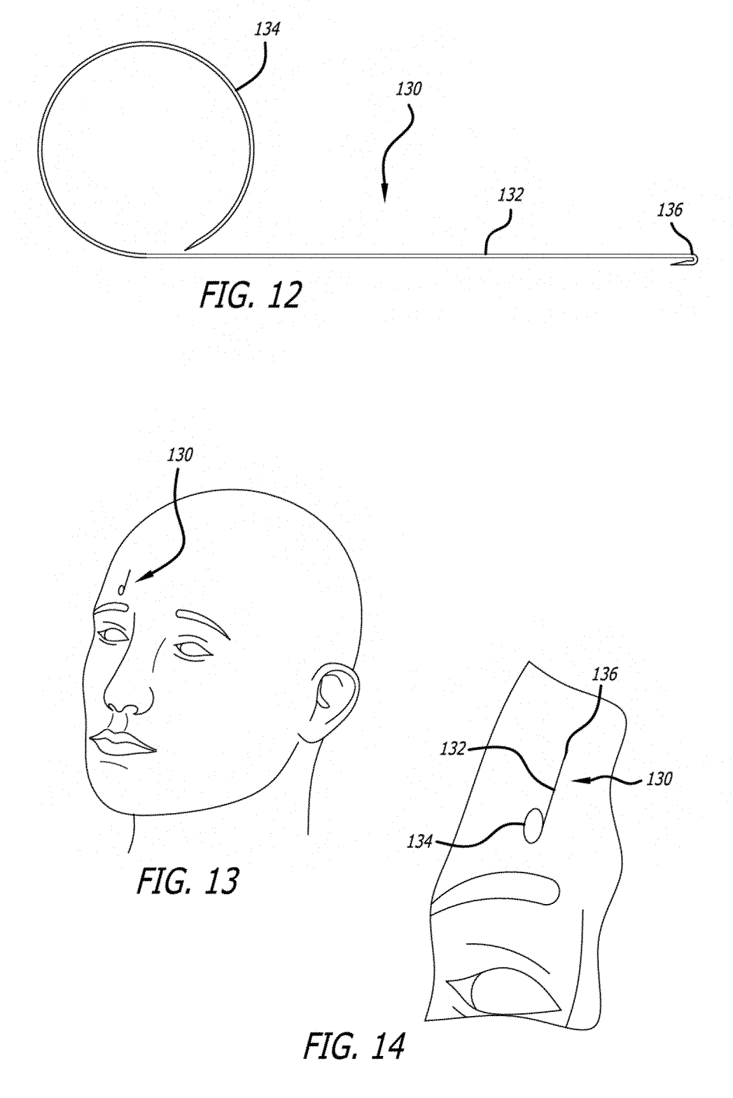

FIG. 12 shows another embodiment of a fastener 130 of the invention. Fastener 130 differs from the other fastener embodiments discussed thus far in that the fastener 130 includes a tissue holder 134 that is laterally displaced from an anchor 136 by a shaft 132 that extends substantially parallel to the bone layer, when implanted. The anchor 136 takes the form of a hook or barb that is used to fix the fastener 130 in position relative to a periosteum.

The tissue holder 134 curls up and away from the periosteum, when implanted, to engage more surface tissue layers such as the connective tissue layer. The positioning of the fastener 130 is depicted in FIGS. 13 and 14, in which the fastener 130 is shown as implanted in the forehead of a patient.

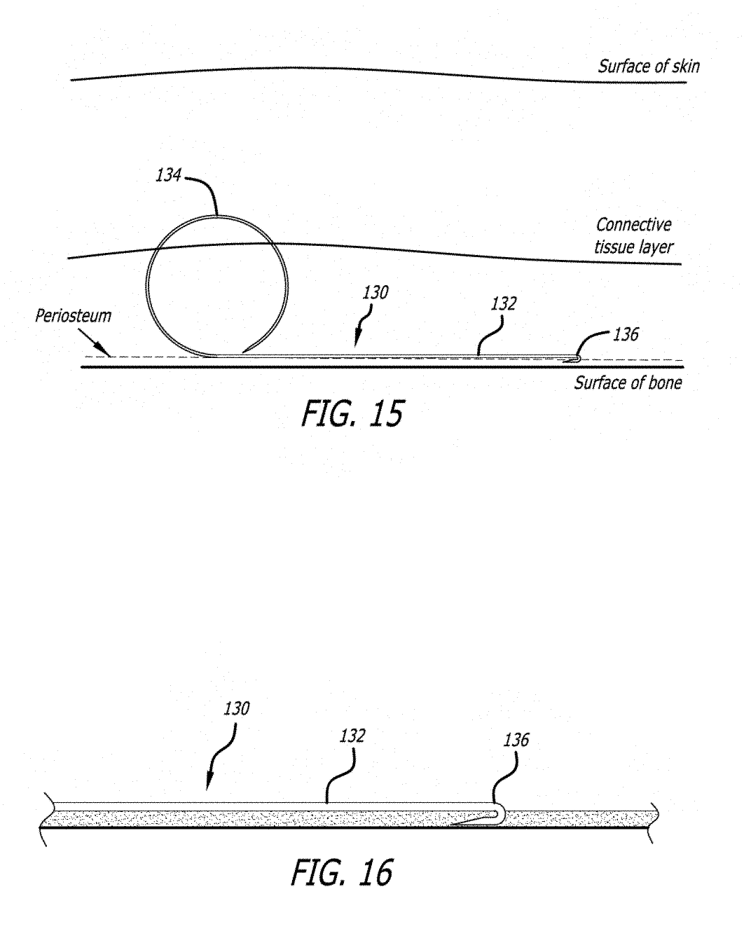

FIG. 15 shows the positioning of the fastener 130 relative to the various tissue layers. The periosteum is shown as a dotted line, just above the surface of the bone. It can be seen that the anchor 136 engages the periosteum, just above the bone layer. The shaft 132 runs substantially parallel to the bone layer. The tissue holder 134 curls away from the periosteum toward the surface of the skin, but remains below the surface. The tissue holder 134 is sized to engage the connective tissue layer without becoming exposed through the surface of the skin. FIG. 16 is a close-up depiction of the anchor 136 engaging the periosteum.

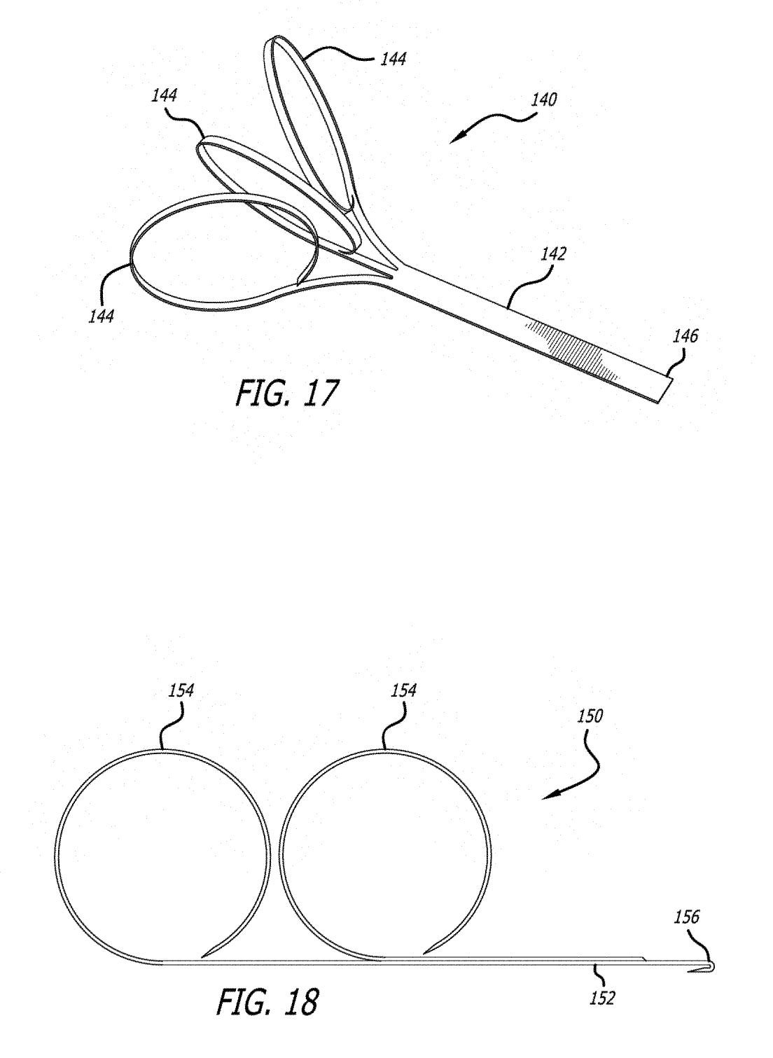

FIG. 17 shows an embodiment 140 of a fastener of the invention. Fastener 140 includes a shaft 142, a tissue holder 144 and an anchor 146. The fastener 140 is distinguished from fastener 130 by the tissue holder or holders 144. Whereas fastener 130 shows a single tissue holder 134, fastener 140 is shown as including multiple tissue holders 144. The fastener 140 is shown in FIG. 17 as including three tissue holders 144 but one skilled in the art will understand that fewer or more may be used. The tissue holders 144 are adjacent to each other and displaced by angling them away from each other.

Another embodiment of a fastener 150 is shown in FIG. 18, which also has multiple tissue holders 155. The tissue holders 155, rather than being adjacent to each other, are spaced apart laterally from each other along shaft 152. Thus one tissue holder 154 is closer to the anchor 156 than the other tissue holder 154. Again, the embodiment of FIG. 18 is shown as having two tissue holders 154 but one skilled in the art will understand that more may be employed without departing from the spirit of the invention. Furthermore, a combination of the tissue holders of FIGS. 17 and 18 could be employed. Such a configuration would include multiple tissue holders that are arranged both adjacent to each other as well as longitudinally spaced.

Fasteners 130, 140 and 150 are all shown as having tissue holders 134, 144, and 154, respectively, that are circular once deployed. These tissue holders are preferably formed from a memory-metal, such as nitinol, and are delivered in a relatively straight configuration and curl to a deployed configuration after implantation, as will be discussed in more detail below.

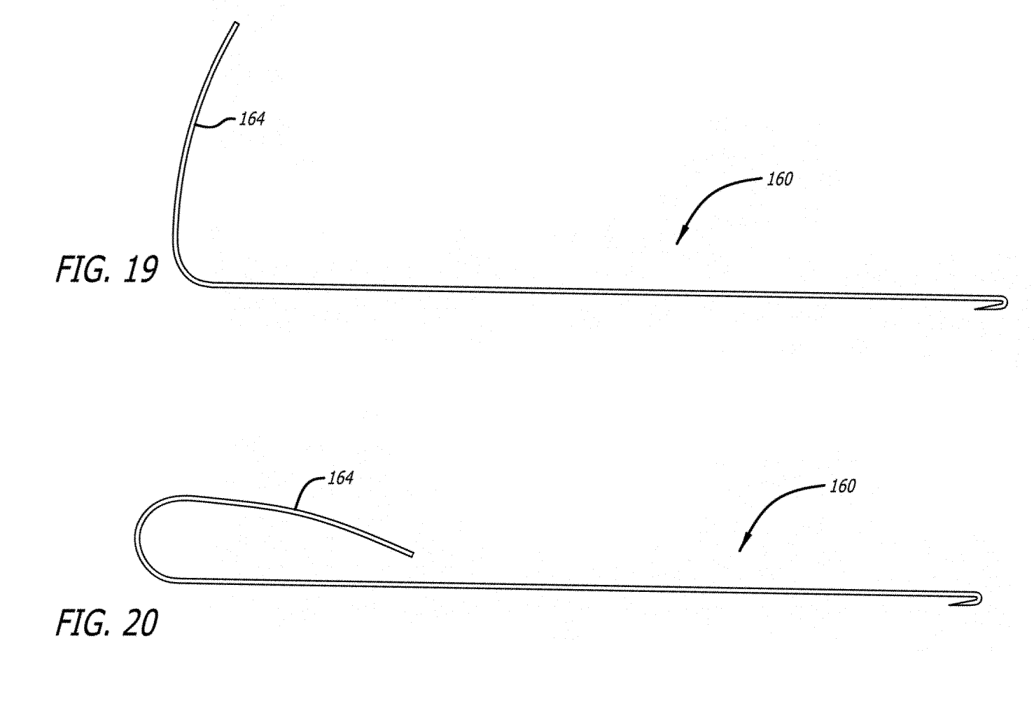

As shown in FIGS. 19 and 20, however, the deployed configuration of the tissue holders is not to be limited to a circular configuration. It may be desired to provide a deeper deployed configuration, such as the fastener 160 shown in FIG. 19, having a complex curve. In FIG. 19, the fastener 160 is partially deployed and the tissue holder 164 extends upward to maximize soft-tissue purchase. In FIG. 20, the fastener 160 has been fully released and has assumed a deployed, fully curved configuration with a low profile.

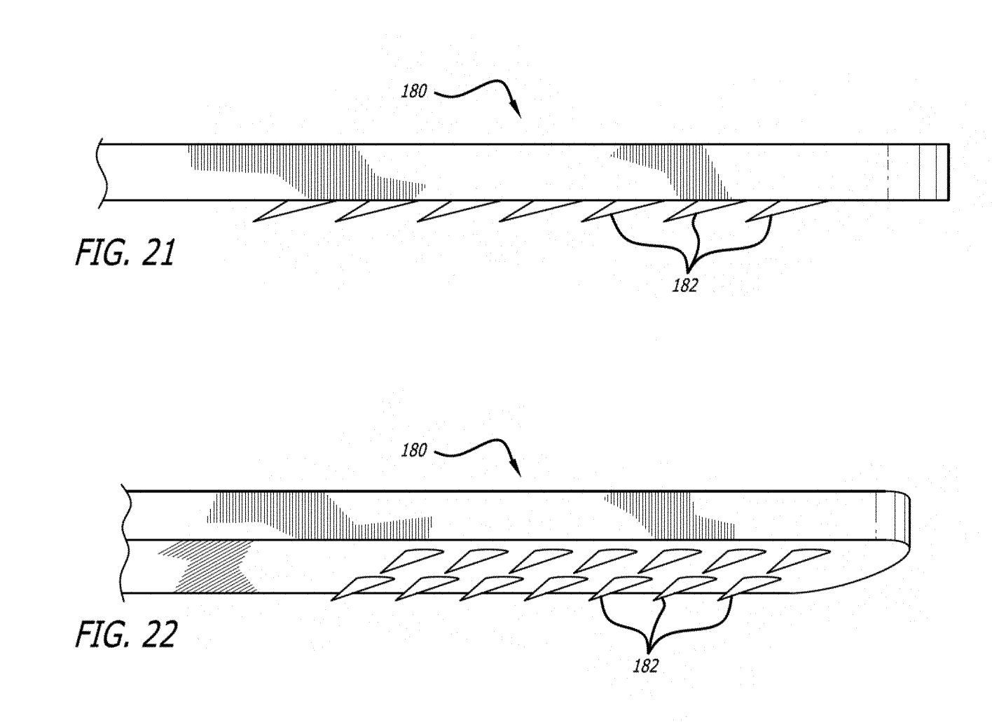

FIGS. 21 and 22 show elevation and perspective views, respectively, of an embodiment of an anchor 180 of the invention that could be used with any of the embodiments 130, 140, 150, or 160 which are positioned with shafts that are substantially perpendicular with the tissue surface to which the fasteners are attached. FIGS. 21 and 22 demonstrate that an anchor 180 may have multiple anchoring points 182. The embodiment shown in FIGS. 21 and 22 have anchor points that angle toward the other end of the fastener, where the tissue holder is located. This is provided as a non-limiting example.

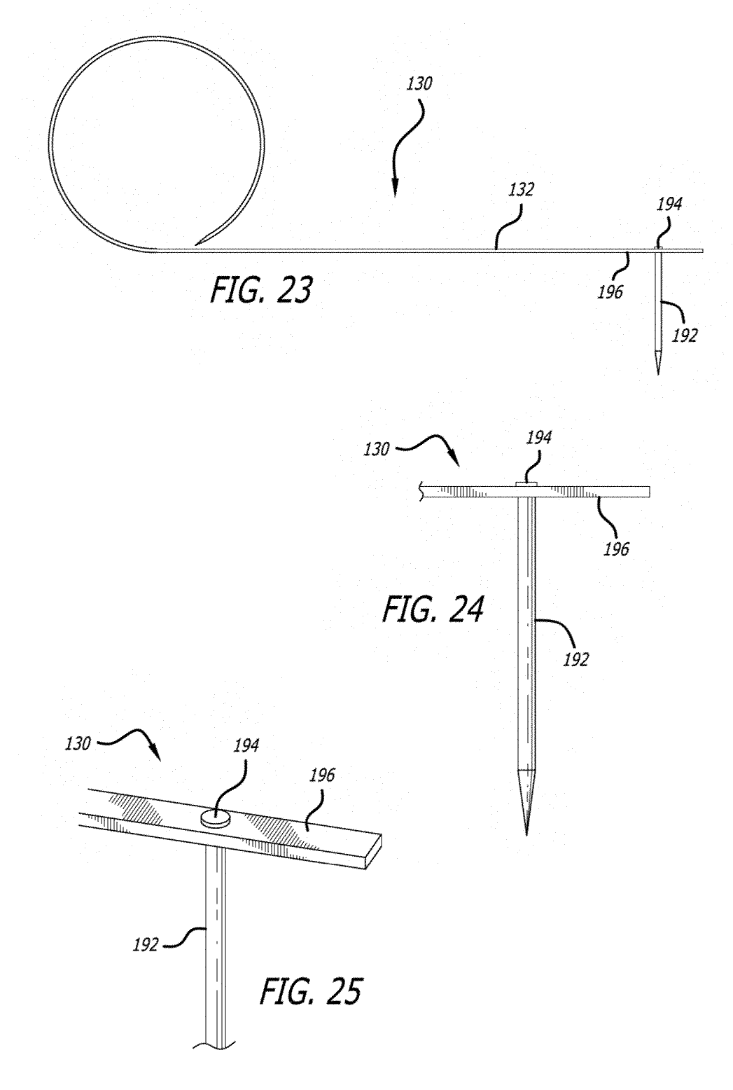

FIGS. 23, 24 and 25 show another embodiment of an anchor 190 that may be used with any of the fastener embodiments 130, 140, 150, or 160. The anchor 190 includes a spike 192 that includes a head 194. The spike passes through the shaft 196 until the head 194 interferes with the shaft 196.

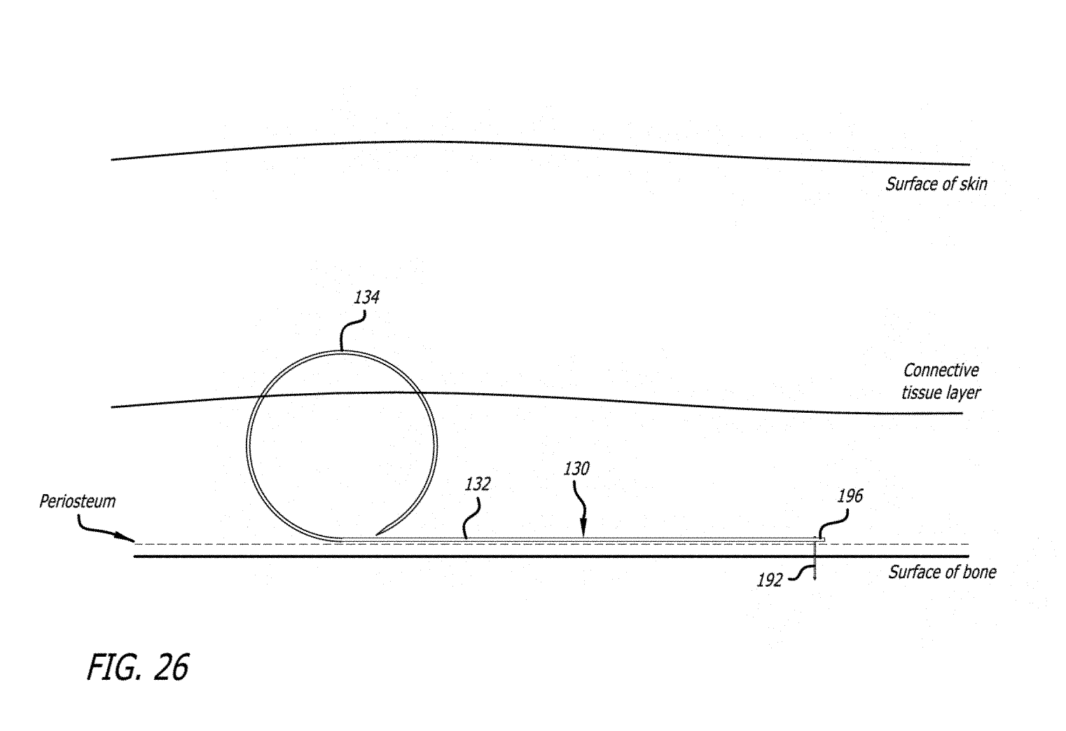

FIG. 26 shows the positioning of a fastener 130 with an anchor 190 relative to the various tissue layers. The periosteum is shown as a dotted line, just above the surface of the bone. It can be seen that the anchor 190 passes through the periosteum into the bone layer. The shaft 132 runs substantially parallel to the bone layer. The tissue holder 134 curls away from the periosteum toward the surface of the skin, but remains below the surface. The tissue holder 134 is sized to engage the connective tissue layer without becoming exposed through the surface of the skin. It is noted that though the anchor 190 appears as a spike, a screw or other similarly-positioned fastener could be used.

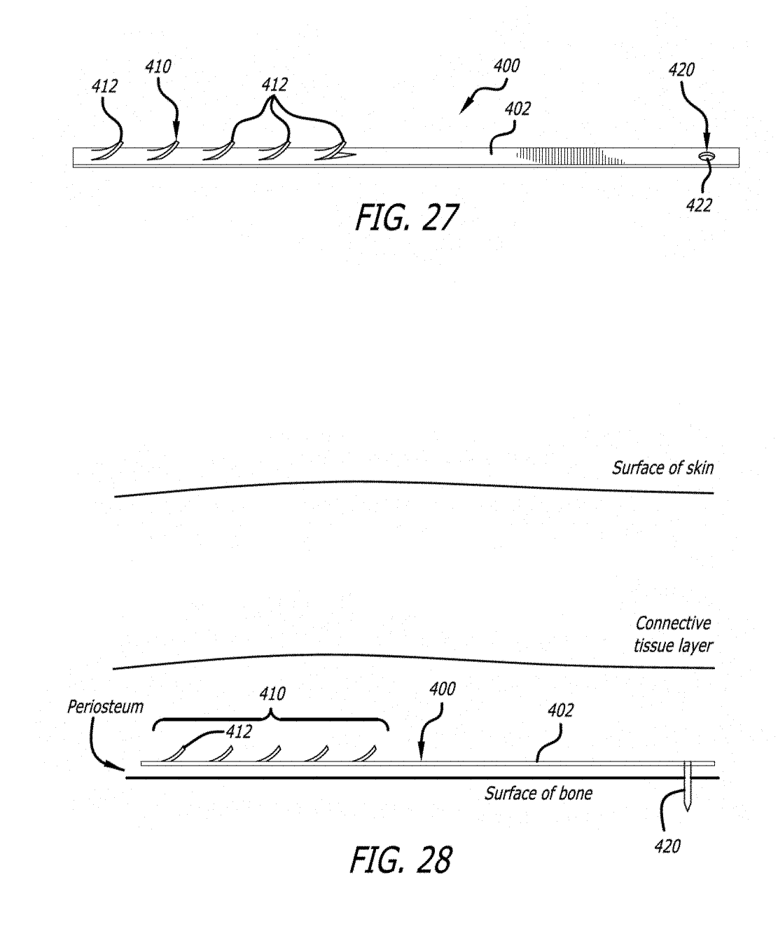

Turning now to FIG. 27 there is shown an embodiment of a fastener 400 having a tissue-holding feature 410 at one end and an anchor feature 420 at a second end. Like embodiments 130, 140, 150, and 160, fastener 400 may be formed of a single, unitary length of material 402, such as a length of Nitinol ribbon, for example.

The tissue-holding feature 410 is shown as one or more barbs 412, which may be cut-out from the length of material 402. In at least one embodiment, the tissue-holding feature 410 has a first configuration and a second configuration. In the first configuration, which is assumed in a delivery device, the tissue-holding feature is straight, such that it may be passed through the delivery device. In the second configuration, the barbs 412 curl outwardly to engage tissue. The second configuration is assumed when the device 400 is released from the delivery device. The length of material 402 may be formed of a memory material, such as Nitinol, and the tissue-holding feature 410 may be biased toward the second configuration.

The anchor feature 420 of embodiment 400 includes a hole 422 for accepting a screw, tack, or other fastener that may be separate or integral with the length of material 402.

FIG. 28 shows the embodiment 400 deployed in a patient. In the configuration shown, the fastener 400 lies on the periosteum and is oriented such that the barbs 412 extend upward into the connective tissue layer. The anchor feature 420 passes through the periosteum into the bone layer.

FIG. 29 shows an embodiment 430 of a fastener that includes a length of material 432 having anchor features 440 on each end. The length of material 432 serves as the tissue holding feature and spreads the compressive force placed on the tissue across the surface of the material 432.

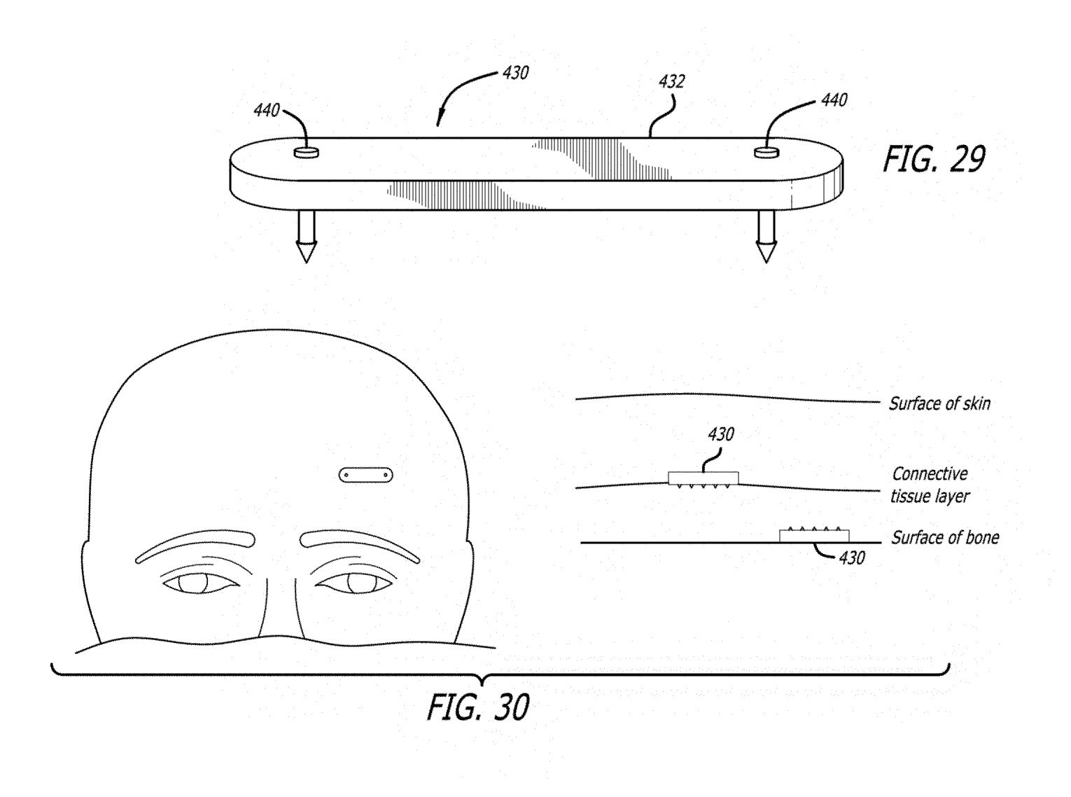

FIG. 30 shows the embodiment 430 deployed in a patient and illustrates that the embodiment 430 could be deployed above or below the connective tissue layer.

Delivery Device

FIG. 32 shows an embodiment of a delivery device of the invention. The delivery device generally includes a firing gun 200 and a disposable tip 300.

The firing gun 200 is a hand-held, preferably spring-powered gun that accepts the disposable tip 300 at its distal end. Alternatively the firing gun could be disposable and incorporate the distal tip. The firing gun may also be powered by compressed gas, electromagnetic mechanisms or other means.

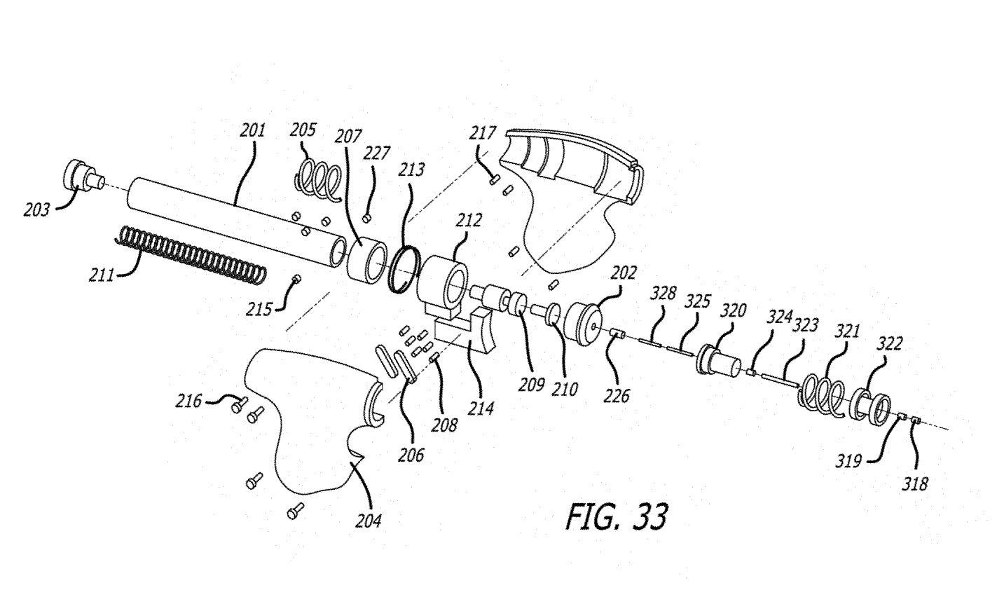

FIG. 33 shows an exploded view of the firing gun 200 and the disposable tip 300. Beginning near the proximal end of the gun 200, there is a chassis 201 that is a rigid tube made of a hard material such as stainless steel. The chassis 201 has an inner lumen that houses a ram spring 211 and prevents the ram spring 211 from deforming outwardly when compressed. The proximal end of the chassis 201 is closed by a ram plug 203.

The distal end of the ram spring 211 includes ram piece 209 distally protected by a metal ram piece 210, which acts against a firing pin 226. Ram piece 209 may thus be constructed of a lighter material, such as plastic or carbon fiber. The pieces 209 and 210 are contained within the chassis 201 with a chassis cap 202. The firing pin 226 extends through an aperture in the cap 202, so that it can impinge against the disposable tip 300, explained in detail below.

The chassis 201 is contained within a gun-shaped housing 204. The housing 204 includes two clam-shell halves that are held together with screws 216 that mate with threaded inserts 217. The housing 204 slides relative to the chassis 201 by a designated amount, in order to provide a safety feature that requires the device to be pressed against tissue to allow the device to be fired. A housing spring 205 acts between the housing 204 and a lock ring 207 to return the housing 204 to a “safe” position when the device is not being pressed against a surface. The lock ring 207 surrounds the chassis 201 and is fixed relative to the chassis 201 with a set screw 227.

Distal of the lock ring 207, and also surrounding the chassis 201, is a slide ring 212. The slide ring 212 slides relative to the chassis 201 to bring an internal groove formed in the slide ring 212 in and out of alignment with ball bearings 215 of the trigger mechanism. The slide ring 212 is connected to a trigger 214 via two linkage bars 206 and spring pins 208.

The trigger holds the ram 209 in a loaded position in which the ram 209 is compressing the ram spring 211, until fired. This is accomplished with the three ball bearings 215 of the trigger mechanism. The ball bearings 215 ride in holes formed in the chassis 201. The diameter of the ball bearings 215 is greater than the thickness of the wall of the chassis 201. Internally, a groove in the ram 209 provides the additional room needed to house the ball bearings 215. The interference created by the ball bearings between the holes of the chassis 201 and the groove of the ram 209 prevents the ram 209 from being able to be propelled through the chassis 201 by the ram spring 211.

As discussed above, the slide ring 212 slides along the exterior of the chassis 201 and has an internal groove. When the internal groove of the slide ring 212 is aligned with the ball bearing holes of the chassis 201, the ball bearings 215 are forced outwardly by the ram 209 into the space provided by the groove of the slide ring 212. The interference between the ram 209 and the chassis 201 is relieved and the ram 209 is freed and propelled by the ram spring 211.

The trigger 214 affects this chain of events. When pulled, the trigger slides the slide ring 212 until the internal groove of the slide ring 212 is in alignment with the ball bearings 215. A trigger spring 213 returns the trigger to a rest position after it is fired.

As discussed above, the housing 204 has a built in feature whereby unless it is advanced by depressed against a surface, the device will not fire. The trigger 214 and the slide ring 212 are constructed and arranged such that the travel limits of the slide ring 212 prevent the internal groove of the slide ring from aligning with the ball bearings 215 unless the housing 204 is advanced a prediscribed distance.

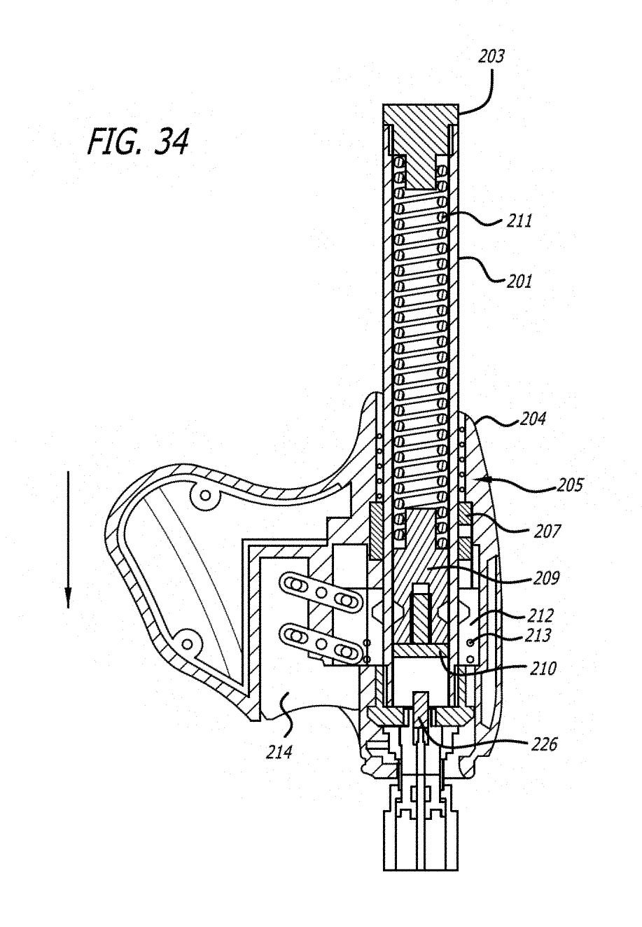

FIG. 34 shows the internal components of the firing mechanism at the moment the device is fired. The housing 204 is advanced by being pressed against a surface, compressing the housing spring 205. The trigger 214 is pulled, against the trigger spring 213, and bringing the internal groove of the slide ring 212 into alignment with the ball bearing holes of the chassis 201.

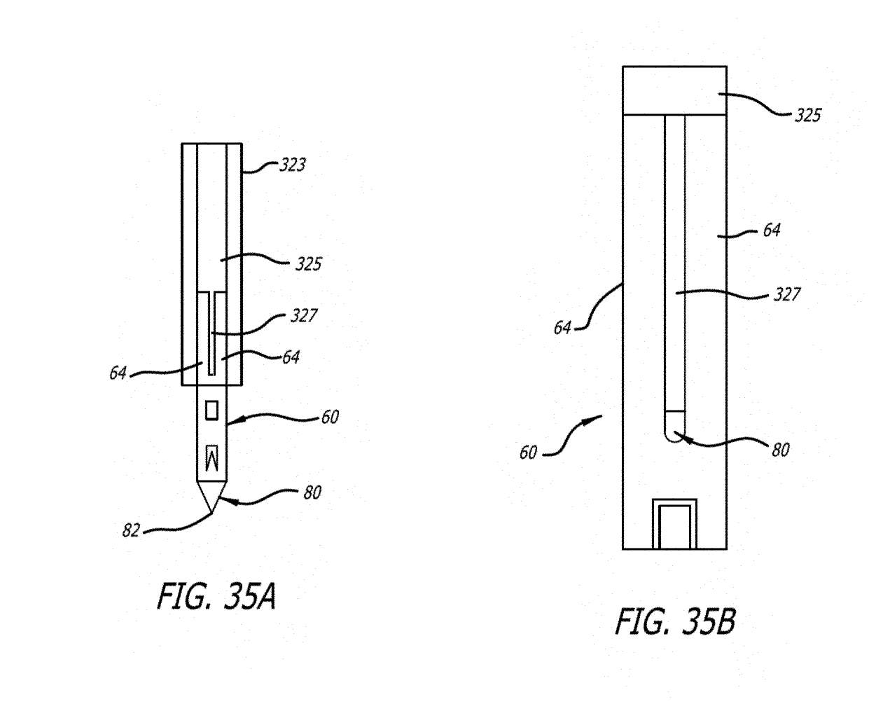

FIGS. 35A and 35B are closeup views of the firing pin 325 as it interacts with a fastener. In this case, the fastener 60 is that of FIG. 5 and has tissue holders 64 in the form of proximal petals. So that the firing pin 325 is acting on the spike 80, and not damaging the petals 64, the firing pin 325 is shaped with a narrower distal end 327 that enters an interior of the fastener 60 and contacts the proximal end of the spike 80.

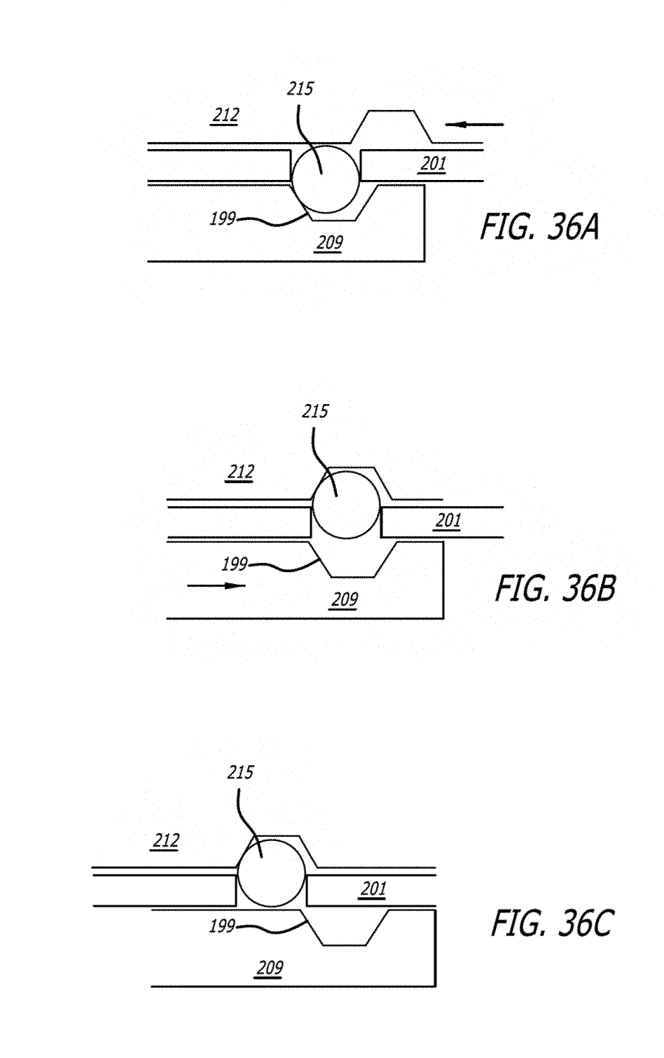

FIGS. 36A-36C show the sequence of events of the firing. In 36A, the ram 209 is unable to be advanced due to the interference between the ram 209 and the chassis 201 created by the ball bearing 215. In 36B, the internal groove of the slide ring 212 is aligned with the ball bearing 215 and the ball bearing 215 is forced into the internal groove of the slide ring 212 by an angled surface 199 of the groove of the ram 209. In 36C, as the interference between the piston 209 and the chassis 201 is no longer being created by the ball bearing 215, the ram 209 is freed and propelled forward.

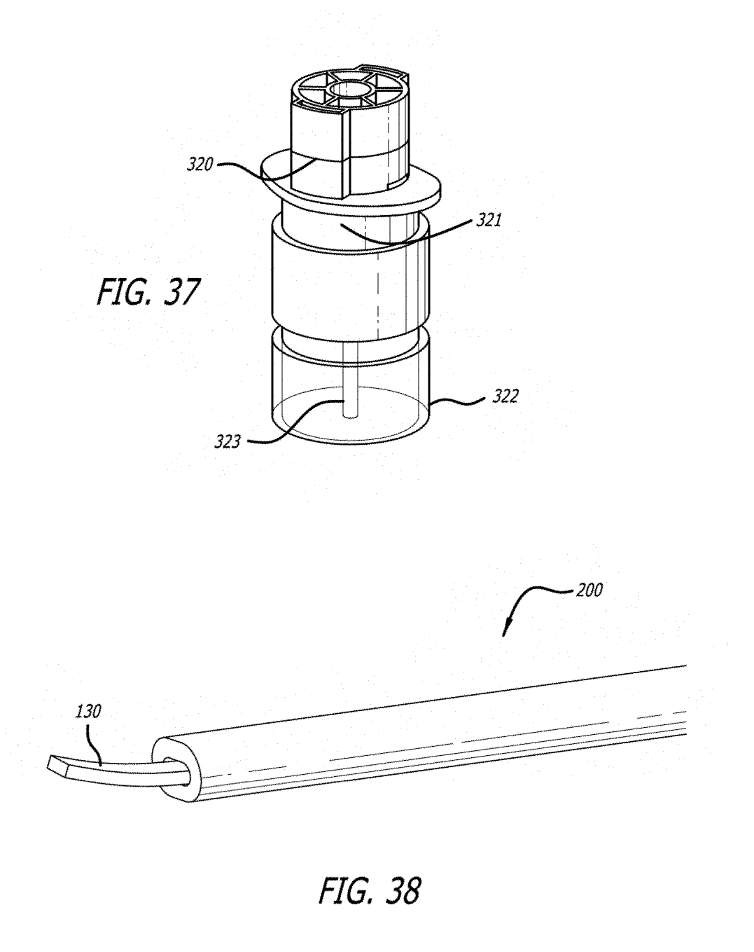

Referring again to FIG. 33, and also to FIG. 37, the disposable tip 300 includes, beginning from a proximal end, an outer firing pin rod 328, an inner firing pin rod 325, a proximal tip component 320, hypotube 324, hypotube 323, a tip spring 321, and a distal tip component 322. Loaded into the distal end of the distal hypotube (or cannula) 323 is the implant and the spike (shown as components 319 and 318 but could be any of the implants and spikes shown and described herein).

The proximal tip component 320 is a plastic piece that is used to attach the tip 300 to the firing gun 200. The proximal tip component 320 includes a mating component that mates with a corresponding mating component at the distal end of the housing 204.

As best seen in FIG. 37, the distal tip component 322 is slidably mated via grooves with the proximal tip component and is biased toward a separated state therewith by a tip spring 321. The tip spring 321 is thus positioned as a shock absorber and allows the physician to press the distal end of the distal component 322 against the patient without undue discomfort. In this regard, the distal component 322 acts as a stabilizer against the skin of the patient. The distal component 322 also acts as an alignment device to aid in orientating the cannula 323 perpendicularly to the surface of the implantation site.

The fastener embodiments 130, 140, 150, and 160 may be used with a different delivery device 200. Referring to FIG. 38, there is shown a delivery device 200 that is in the form of a low-profile catheter shaped to slideably house the fastener. Fastener 130 is used as an example in FIG. 38.

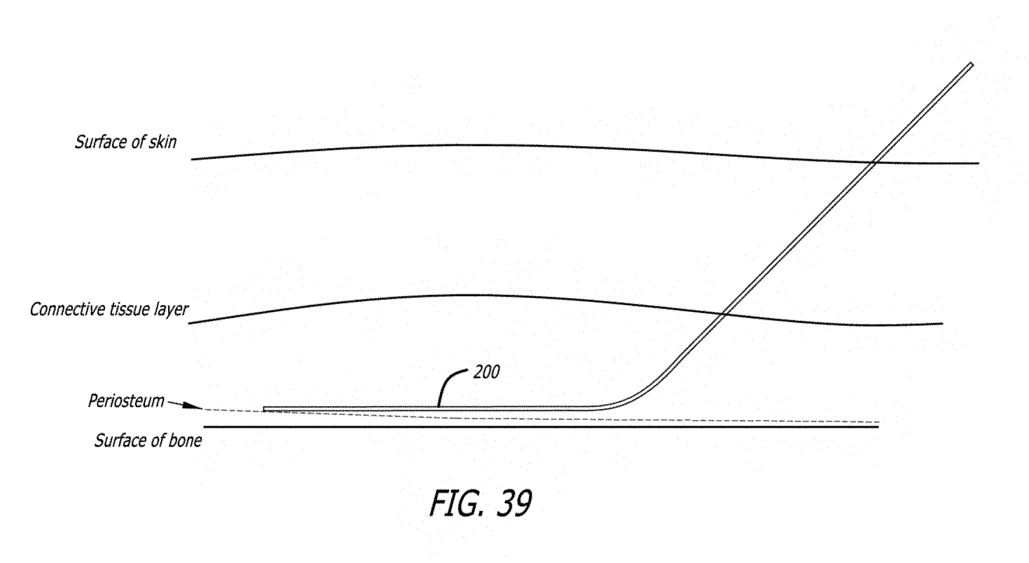

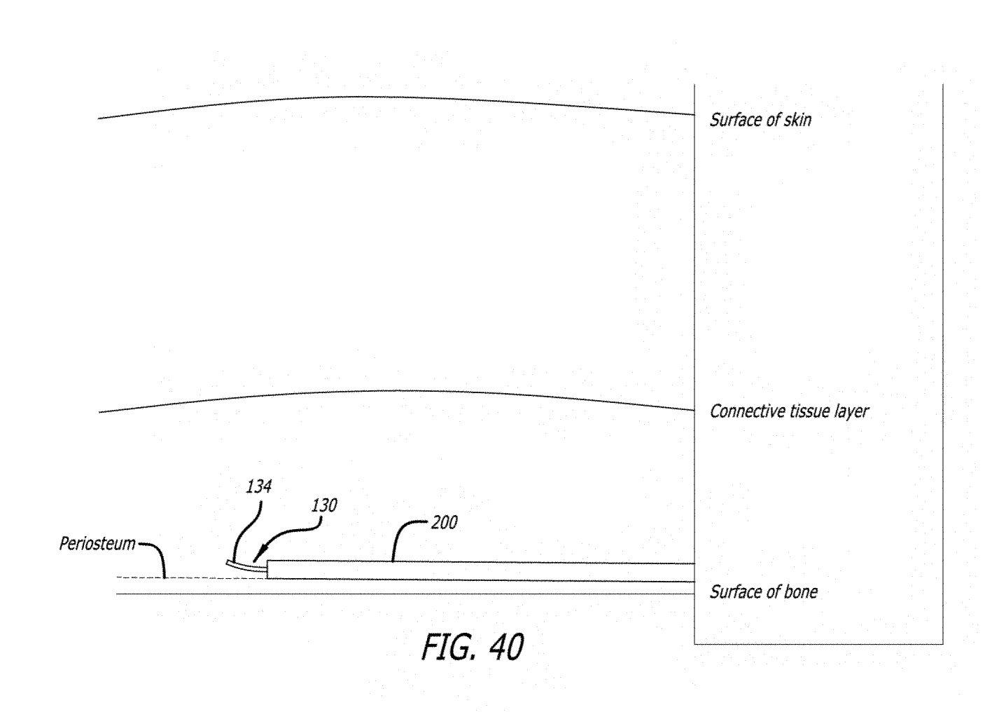

FIG. 39 shows the first in a series of delivery steps utilizing the delivery device 200. As can be seen, the delivery device 200 is routed through an incision along the periosteum until a target location is reached. At this point, as best seen in FIG. 40, the fastener 130 is ejected from the distal end of the delivery device 200. The distal end of the fastener 130 is the tissue holder 134.

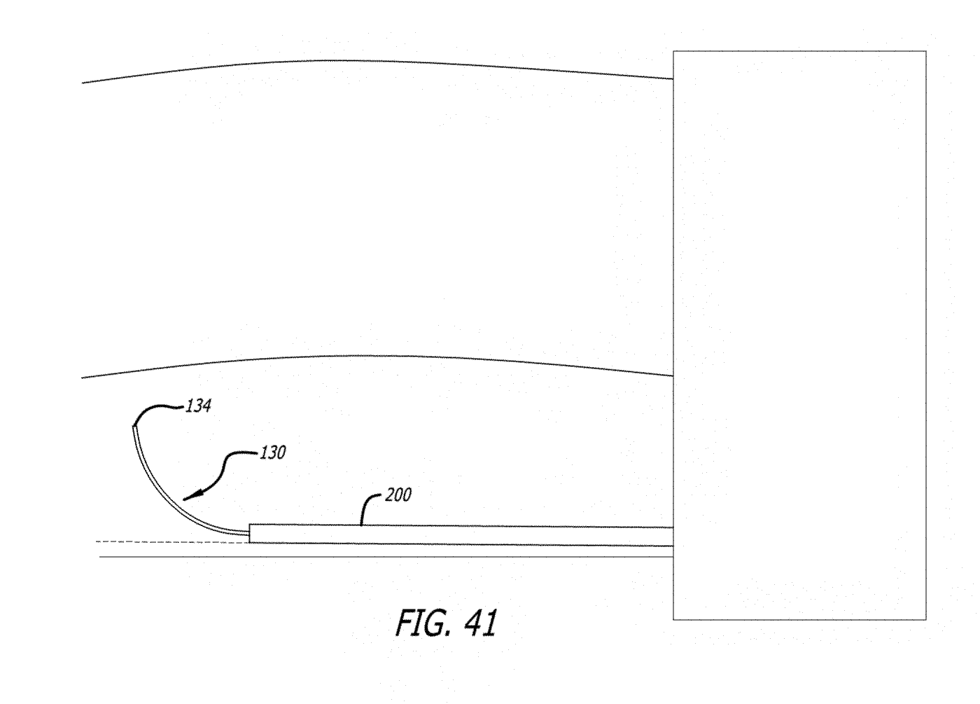

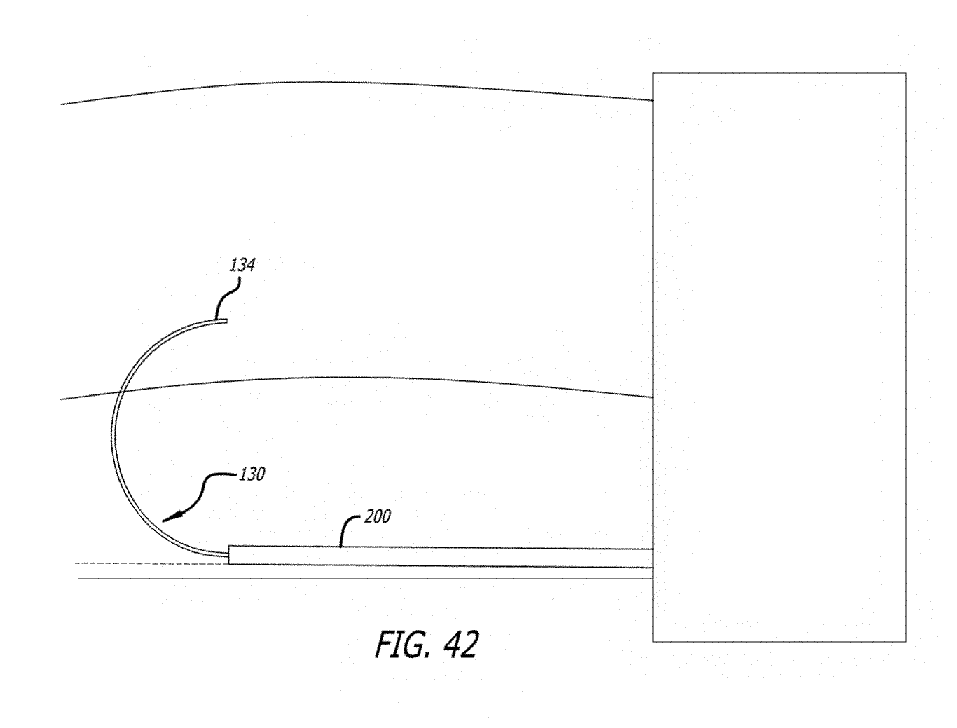

FIGS. 41 and 42 show the tissue holder 134 curling upwardly toward the tissue surface as the fastener 130 continues to be ejected from the delivery device 200. As the tissue holder 134 curls, the holder 134 engages and holds onto tissue.

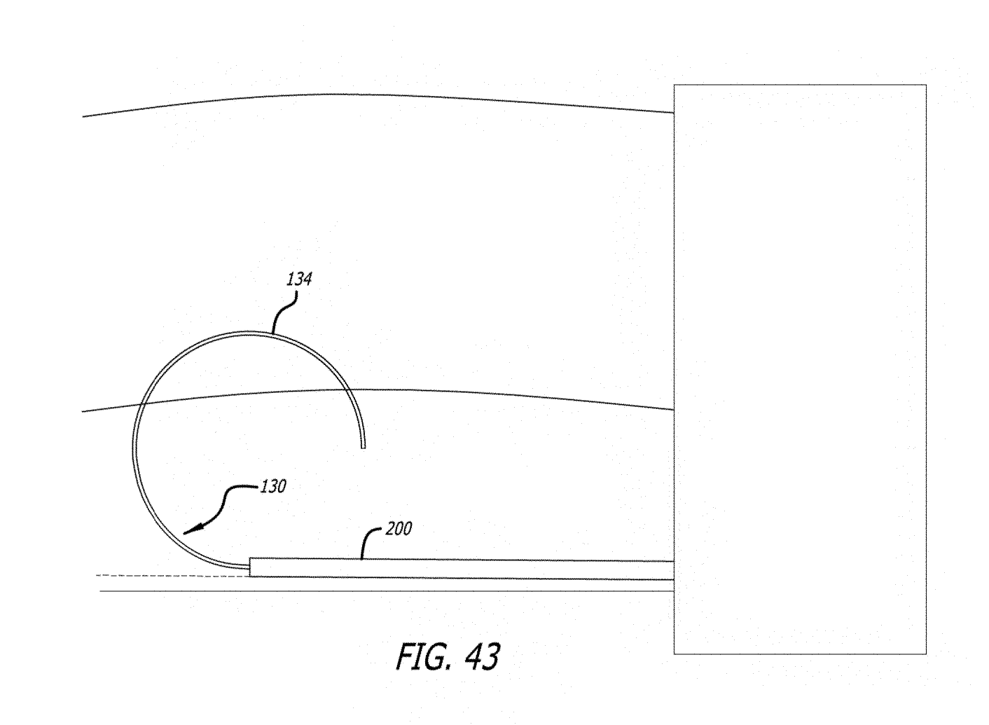

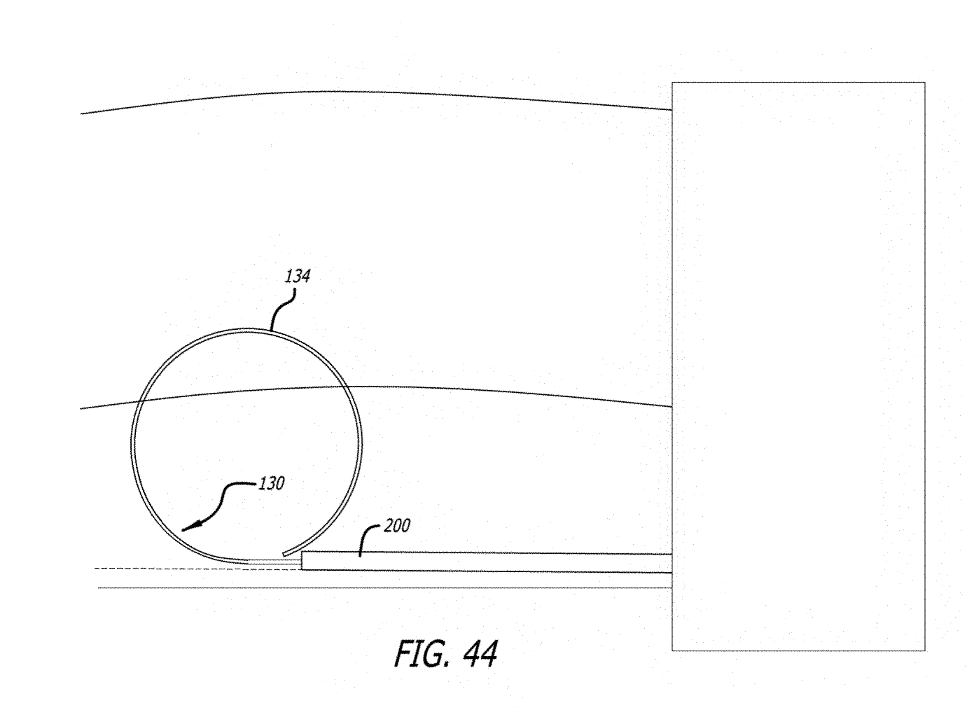

FIGS. 43 and 44 show the tissue holder 134 curling back toward the delivery device 200. One skilled in the art will realize that different degrees of curling could be utilized and may vary depending on the location and type of tissue being engaged.

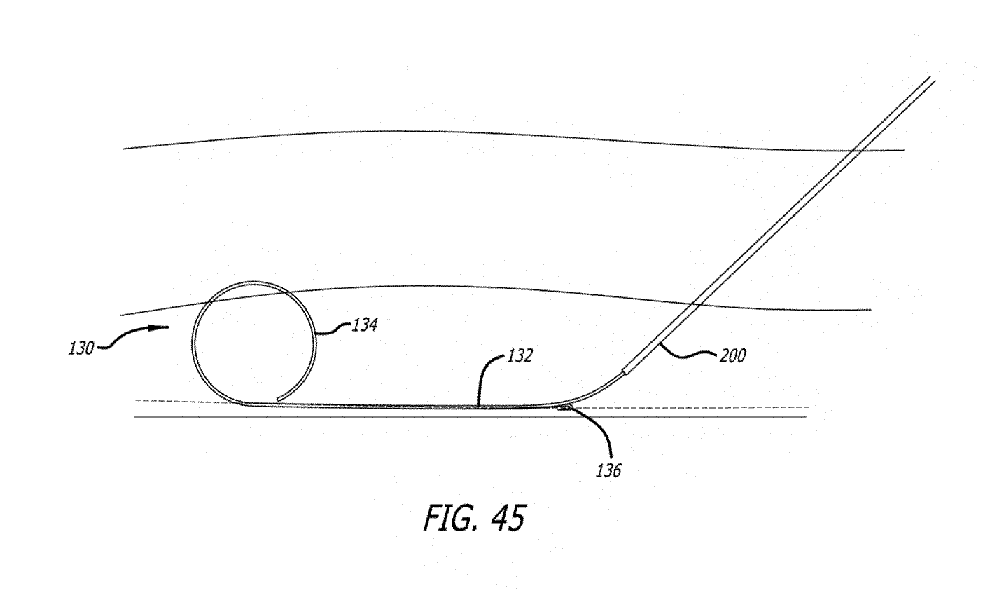

In FIG. 45, the tissue holder 134 has been fully deployed and the delivery device 200 has been retracted from the target site, fully exposing the shaft 132 and the anchor 136. Due to the elastic forces the surface skin layers place on the tissue holder 134, the anchor 136 naturally engages the periosteum. However, downward force may be placed on the anchor 136 to enhance engagement of the periosteum.

Though the delivery device 200 is shown as deploying a fastener 130 with the tissue holder 134 emerging first, it is contemplated that the fastener 130, 140, 150, or 160 could be loaded into the delivery device 200 in a reverse order such that the anchor 136, etc., emerges first and engages the periosteum immediately while the tissue holder 134, etc., emerges last.

Operation Dynamics

Through computation and experimentation, it has been determined that given the relatively hard bone of the human skull, a sharp, solid pin is preferred to initiate penetration. However, fasteners may also be configured as a sharpened hypotube without the central pin. For any given implant configuration, including factors such as fastener diameter, length, sharpness, tissue characteristics, and the like, there is minimum energy threshold that is required to drive the fastener effectively into the bone to the desired depth. Furthermore, various types of bone vary in their thickness and hardness, which effects the energy necessary for implantation, as well as the strength and features needed in the implant itself.

The embodiments of the driving devices described herein use a spring. The potential energy stored in the ram spring is converted quickly to kinetic energy when the spring is released. That kinetic energy is transferred, in turn, to the fastener. Though spring energy is used in the embodiments described herein, one skilled in the art will understand that pneumatic, ultrasound, electric, or a variety of other energy sources, could be used to achieve the results of the invention.

It has been determined that if one exceeds the minimum energy level for implantation, the depth that the fastener is driven into the skull can be controlled by the length of travel of the piston/firing pin. Because the fastener has very little mass, and therefore very little momentum, the fastener will not continue to travel deeper into the tissue once the energy source is stopped or otherwise isolated from the fastener. Other means of controlling fastener depth are possible, including controlling the total energy applied to the fastener, or the depth of a pre-drilled hole.

Failure to achieve the minimum energy level typically results in a deformation of the fastener as it is being driven into the skull, or at a minimum results in an fastener that is delivered to a sub-optimal depth. It is thought that a failure to achieve the minimum energy level results in a reduced fastener velocity, which gives the fastener additional time to deform upon entry into the skull. At normal speeds, during a perfectly inelastic collision, an object struck by a projectile will deform, and this deformation will absorb most or all of the force of the collision. Viewed from a conservation of energy perspective, the kinetic energy of the projectile is changed into heat and sound energy, as a result of the deformations and vibrations induced in the struck object. However, these deformations and vibrations cannot occur instantaneously. A high-velocity collision (an impact) does not provide sufficient time for these deformations and vibrations to occur. Thus, the struck material behaves as if it were more brittle than it would otherwise be, and the majority of the applied force goes into fracturing the material.

This minimum energy is thus factored into the selection of the spring strength, the travel distance and mass of the metal ram 210 and ram 209. The spring must be strong enough, and the travel of the ram 210 and 209 must be long enough, such that speed of the ram 210 and 209 achieves a minimum velocity prior to impacting the firing pin 226. Additionally the mass of 209 and 210 must be such that the kinetic energy of the spring is sufficient to accelerate 209 and 210 to a critical velocity. This mass also plays a role in the amount of momentum that is transferred to the firing pin. Alternatively, the ram 210 and 209 and the firing pin 226 could comprise a single piece, in which case the pin 226 would be propelled to a minimum velocity by the ram spring 211 prior to impacting the fastener assembly.

Assuming the minimum fastener velocity is achieved, the depth of the fastener can be controlled in a variety of ways. As stated above, one way to control depth is to limit the travel of the firing pin. As the fastener has very little mass, the fastener will not continue to travel beyond the travel of the firing pin. It is also possible to control fastener depth or pin travel by precisely controlling the energy delivered to the fastener or pin.

It is also possible to control firing pin travel using other methods, some of which allow a lighter ram spring to be used. For example, predrilling a hole for the fastener allows the fastener to be driven using a lighter driving force. Both the depth of the pre-drilled hole, as well as the diameter of the pre-drilled hole relative to the diameter of the fastener can be varied to control the resulting fastener depth.

Although the invention has been described in terms of particular embodiments and applications, one of ordinary skill in the art, in light of this teaching, can generate additional embodiments and modifications without departing from the spirit of or exceeding the scope of the claimed invention. Accordingly, it is to be understood that the drawings and descriptions herein are proffered by way of example to facilitate comprehension of the invention and should not be construed to limit the scope thereof.

What is claimed is:

1. A method for relocating a skin and/or soft tissue layer relative to an adjacent bone layer comprising:
shifting a skin and/or soft tissue layer relative to an adjacent bone layer from an original position to a shifted position without mechanically separating muscle or connective tissue associated with said skin and/or soft tissue layer from the skin layer, underlying tissue or bone;
holding said skin and/or soft tissue layer in said shifted position while driving a fastener through said skin layer into said bone layer a desired depth, with features on the fastener to prevent said skin and/or soft tissue layer from reassuming said original position;
wherein driving said fastener through said skin and/or soft tissue layer into said bone layer comprises transferring a minimum amount of energy to said fastener.

2. The method of claim 1 wherein transferring a minimum amount of energy to said fastener comprises accelerating said fastener to a minimum velocity.

3. The method of claim 2 wherein said minimum velocity is determined by at least one of a size of said fastener and a type of anatomic material being targeted.

4. The method of claim 2 wherein said minimum velocity is determined by a size of a predrilled hole in said bone relative to a size of said fastener.

5. The method of claim 4 wherein said size of said predrilled hole comprises a depth of said predrilled hole.

6. The method of claim 4 wherein said size of said predrilled hole comprises a width of said predrilled hole.

7. The method of claim 1 wherein transferring a minimum amount of energy to said fastener is accomplished by accelerating said fastener with a firing pin.

8. The method of claim 7 wherein said depth the fastener is driven into said bone layer is controlled by a travel length of the firing pin.

9. An implant for use in anchoring a skin and/or soft tissue layer in a shifted position to a bone layer thereby overcoming lateral and axial forces imparted by said skin and/or soft tissue layer on said implant comprising:
a fastener having a hollow shaft with a distal end and a proximal end, said hollow shaft including:
an anchoring feature proximate said distal end; and,
a tissue-holding feature proximate said proximal end;
a spike housed within said hollow shaft and protruding from a distal end thereof, said spike including a sharpened tip.

10. The implant of claim 9 wherein said tissue-holding feature comprises at least one petal that flares outwardly upon deployment.

11. The implant of claim 10 wherein said at least one petal comprises a memory metal.

12. The implant of claim 9 wherein said anchoring feature comprises at least one barb that flares outwardly upon deployment.

13. The implant of claim 9 wherein said distal end of said hollow shaft comprises a bevel that aligns with a surface of said sharpened tip.

14. The implant of claim 9 wherein said sharpened tip comprises a flange that has a diameter greater than a diameter of said hollow shaft.

15. A device for use in anchoring a skin and/or soft tissue layer in a shifted position relative to a bone layer comprising:
a firing gun including;
a handle
a firing mechanism disposed within said handle and including a firing pin and a trigger mechanism;
a disposable tip removably attached to a distal end of said firing gun and including:
a proximal tip component that removably attaches the disposable tip to the distal end of the firing gun;
a distal tip component distally attached to said proximal tip component;
an implant carried within said distal tip component;
wherein when said trigger mechanism is pulled:
said firing mechanism is activated, thereby transferring energy to said firing pin;
said firing pin in turn accelerates said implant to at least a minimum velocity, thereby driving said implant into an implantation site.

16. The device of claim 15 wherein firing mechanism comprises a spring.

17. The device of claim 15 wherein said proximal tip component is slidingly attached to said distal tip component.

18. The device of claim 17 further comprising a spring disposed between said proximal tip component and said distal tip component biasing the components into an expanded configuration.

19. The device of claim 15 further comprising a safety feature requiring said firing gun to be compressed against a surface in order to activate said triggering mechanism.

20. The device of claim 15 wherein said implant comprises a spike housed within a fastener and having a sharpened tip protruding distally therefrom.

21. A device for use in anchoring a tissue layer in a shifted position comprising:
a length of material having a first end and a second end;
said first end including a tissue-holding feature that has a first configuration and a second configuration, wherein said first configuration is straight and said second configuration is curled;
said second end including an anchor usable to fasten said device to a feature such that tissue held by said tissue-holding feature is maintained in a shifted position.

22. The device of claim 21 wherein when said length of material is released from a delivery device, said tissue-holding feature assumes said second configuration from said first configuration.

23. The device of claim 21 wherein said tissue-holding feature comprises a plurality of petals.

24. The device of claim 21 wherein said second configuration comprises at least one curl in said first end to form said tissue-holding feature.

25. The device of claim 21 wherein said anchor comprises at least one barb.

26. The device of claim 21 wherein said anchor comprises threads.

27. The device of claim 21 wherein said anchor comprises a fastener passing through said second end.

28. The device of claim 21 wherein said tissue-holding feature comprises a plurality of cut-out barbs.
